(19) United States
(12) Patent Application Publication
(10) Pub. No.: US 2007/0155997 A1
(43) Pub. Date: Jul. 5, 2007

(54) Organic compositions

(76) Inventors: Bo Li, San Jose, CA (US); Kreisler Lau, Sunnyvale, CA (US); Paul Apen, San Francisco, CA (US)

Correspondence Address: BINGHAM MCCUTCHEN LLP, Three Embarcadero Center, San Francisco, CA, 94111-4067, US

(21) Appl. No.: 10/536,884

(22) PCT Filed: Dec. 31, 2003
(86) PCT No.: PCT/US03/41533

§ 371 Date: Jun. 29, 2006

Related U.S. Application Data
(60) Provisional application No. 60/436,927, filed on Dec. 30, 2002.

(51) Int. Cl. C07C 13/615

(52) U.S. Cl. 585/21

(57) ABSTRACT

Compositions and methods of forming and using those compositions are provided herein where the composition comprises at least one oligomer or polymer of Formula I wherein E is a cage compound; each Q is the same or different and selected from aryl, branched aryl, and substituted aryl wherein the substituents include hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl; A is substituted or unsubstituted aryl with substituted or unsubstituted arylalkynyl group (substituents include hydrogen, halogen, alkyl, phenyl or substituted aryl; and aryl includes phenyl, biphenyl, naphthyl, terphenyl, anthracenyl, polyphenylene, polyphenylene ether, or substituted aryl); h is from 0 to 10; i is from 0 to 10; j is from 0 to 10; and w is 0 or 1.

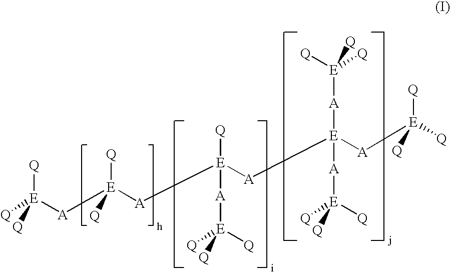

### FIELD OF THE SUBJECT MATTER

The field of the subject matter disclosed herein is related to a composition, and in particular, tetrasubstituted adamantane derivatives, and oligomers or polymers thereof linked via unsubstituted or substituted phenyl units, to a process for its preparation and to its use, inter alia as a dielectric or insulation material in microelectronic components.

### BACKGROUND

Dielectrics are widely used in the semiconductor industry, e.g. as insulation material between conductive lines, such as integrated circuits, microchips, multichip modules, laminated circuit boards or other microelectronic components.

The advances in the semiconductor industry rest on the continuing development of new generations of integrated circuits that display a higher capacity and functionality at the same time as the dimensions become smaller. Since the conductive lines thus have to be ever finer and more densely packed, the capacitance between the neighbouring conductive lines increases, which is associated with a series of disadvantages, such as increased current consumption, longer signal delay time and more crosstalk.

Methods used to deposit dielectric materials may be divided into two categories: spin-on deposition (hereinafter SOD) and chemical vapor deposition (hereinafter CVD). Efforts to develop lower dielectric constant materials include altering the chemical composition (organic, inorganic, blend of organic/inorganic) or changing the dielectric matrix (porous, non-porous). Table I summarizes the development of several materials having dielectric constants ranging from 2.0 to 3.5. (PE=plasma enhanced; HDP=high-density plasma) However, many of these dielectric materials and matrices disclosed in the publications shown in Table 1 fail to exhibit many of the necessary or optimal physical and chemical properties needed for low k dielectric materials, such as higher mechanical stability, high thermal stability, high glass transition temperature, high modulus or hardness, while at the same time still being able to be processed on to a substrate, wafer, or other surface. Therefore, it may be useful to investigate other compounds and materials that may be used as dielectric materials and layers, even though these compounds or materials may not be currently contemplated as dielectric materials in their present form.

TABLE I

| | DEPOSITION | DIELECTRIC | |
|---|---|---|---|
| MATERIAL | METHOD | CONSTANT (k) | REFERENCE |
| Fluorinated silicon oxide | PE-CVD; | 3.3-3.5 | U.S. Pat. No. 6,278,174 |
| (SiOF) | HDP-CVD | | |
| Hydrogen | SOD | 2.0-2.5 | U.S. Pat. Nos. 4,756,977; 5,370,903; and |
| Silsesquioxane (HSQ) | | | 5,486,564; International Patent Publication |
| | | | WO 00/40637; E. S. Moyer et al., |
| | | | “Ultra Low k Silsesquioxane Based Resins”, |
| | | | Concepts and Needs for Low Dielectric |
| | | | Constant <0.15 μm Interconnect Materials: |
| | | | Now and the Next Millennium, Sponsored by |
| | | | the American Chemical Society, pages 128- |
| | | | 146 (Nov. 14-17, 1999) |
| Methyl Silsesquioxane | SOD | 2.4-2.7 | U.S. Pat. No. 6,143,855 |
| (MSQ) | | | |
| Polyorganosilicon | SOD | 2.5-2.6 | U.S. Pat. No. 6,225,238 |
| Fluorinated Amorphous | HDP-CVD | 2.3 | U.S. Pat. No. 5,900,290 |
| Carbon (a-C:F) | | | |
| Benzocyclobutene(BCB) | SOD | 2.4-2.7 | U.S. Pat. No. 5,225,586 |
| Polyarylene Ether(PAE) | SOD | 2.4 | U.S. Pat. Nos. 5,986,045; 5,874,51.6; and |
| | | | 5,658,994 |
| Parylene (N and F) | CVD | 2.4 | U.S. Pat. No. 5,268,202 |
| Polyphenylenes | SOD | 2.6 | U.S. Pat. Nos. 5,965,679 and 6,288,188B1; |
| | | | and Waeterloos et al., “Integration Feasibility |
| | | | of Porous SiLK Semiconductor Dielectric”, |
| | | | Proc. Of the 2001 International Interconnect |
| | | | Tech. Conf., pp. 253-254 (2001). |
| Thermosettable | SOD | 2.3 | International Patent Publication WO |
| benzocyclobutenes, | | | 00/31183 |
| polyarylenes, | | | |
| thermosettable | | | |
| perfluoroethylene | | | |
| monomer | | | |
| Poly(phenylquinoxaline), | SOD | 2.3-3.0 | U.S. Pat. Nos. 5,776,990; 5,895,263; |
| organic polysilica | | | 6,107,357; and 6,342,454; and US Patent |
| | | | Publication 2001/0040294 |
| Organic polysilica | SOD | Not reported | U.S. Pat. No. 6,271,273 |
| Organic and inorganic | SOD | 2.0-2.5 | Honeywell U.S. Pat. No. 6,156,812 |
| Materials | | | |
| Organic and inorganic | SOD | 2.0-2.3 | Honeywell U.S. Pat. No. 6,171,687 |
| Materials | | | |
| Organic materials | SOD | Not reported | Honeywell U.S. Pat. No. 6,172,128 |
| Organic | SOD | 2.12 | Honeywell U.S. Pat. No. 6,214,746 |
| Organic and inorganic | SOD | Not reported | Honeywell U.S. Pat. No. 6,313,185 |
| materials | | | |
| Organosilsesquioxane | CVD, SOD | <3.9 | Honeywell WO 01/29052 |
| Fluorosilsesquioxane | CVD, SOD | <3.9 | Honeywell U.S. Pat. No. 6,440,550 |
| Organic and inorganic | SOD | ≦2.5 | Honeywell U.S. Pat. No. 6,380,270 |
| materials | | | |
| Organic materials | — | <3.0 | Honeywell U.S. Pat. No. 6,380,347 |
| Cage based structure | SOD | <2.7 | Honeywell Serial 10/158513 |
| | | | filed May 30, 2002 |
| Cage based structure | SOD | <3.0 | Honeywell Serial 10/158548 |
| | | | filed May 30, 2002 |

Unfortunately, numerous organic SOD systems under development with a dielectric constant between 2.0 and 3.5 suffer from certain drawbacks in terms of mechanical and thermal properties as described above; therefore a need exists in the industry to develop improved processing and performance for dielectric films in this dielectric constant range.

Reichert and Mathias describe compounds and monomers that comprise adamantane molecules, which are in the class of cage-based molecules and are taught to be useful as diamond substitutes. (Polym, Prepr. (Am. Chem. Soc., Div. Polym. Chem.), 1993, Vol. 34 (1), pp. 495-6; Polym, Prepr. (Am. Chem. Soc., Div. Polym. Chem.), 1992, Vol. 33 (2), pp. 144-5; Chem. Mater., 1993, Vol. 5 (1), pp. 4-5; Macromolecules, 1994, Vol. 27 (24), pp. 7030-7034; Macromolecules, 1994, Vol. 27 (24), pp. 7015-7023; Polym, Prepr. (Am. Chem. Soc., Div. Polym. Chem.), 1995, Vol. 36 (1), pp. 741-742; 205^{th} ACS National Meeting, Conference Program, 1993, pp. 312; Macromolecules, 1994, Vol. 27 (24), pp. 7024-9; Macromolecules, 1992, Vol. 25.(9), pp. 2294-306; Macromolecules, 1991, Vol. 24 (18), pp. 5232-3; Veronica R. Reichert, PhD Dissertation, 1994, Vol. 55-06B; ACS Symp. Ser.: Step-Growth Polymers for High-Performance Materials, 1996, Vol. 624, pp. 197-207; Macromolecules, 2000, Vol. 33 (10), pp. 3855-3859; Polym, Prepr. (Am. Chem. Soc., Div. Polym. Chem.), 1999, Vol. 40 (2), pp. 620-621; Polym, Prepr. (Am. Chem. Soc., Div. Polym. Chem.), 1999, Vol. 40 (2), pp. 577-78; Macromolecules, 1997, Vol. 30 (19), pp. 5970-5975; J. Polym. Sci, Part A: Polymer Chemistry, 1997, Vol. 35 (9), pp. 1743-1751; Polym, Prepr. (Am. Chem. Soc., Div. Polym. Chem.), 1996, Vol. 37 (2), pp. 243-244; Polym, Prepr. (Am. Chem. Soc., Div. Polym. Chem.), 1996, Vol. 37 (1), pp. 551-552; J. Polym. Sci., Part A: Polymer Chemistry, 1996, Vol. 34 (3), pp. 397-402; Polym, Prepr. (Am. Chem. Soc., Div. Polym. Chem.), 1995, Vol. 36 (2), pp. 140-141; Polym, Prepr. (Am. Chem. Soc., Div. Polym. Chem.), 1992, Vol. 33 (2), pp. 146-147; J. Appl. Polym. Sci., 1998, Vol. 68 (3), pp. 475-482). The adamantane-based compounds and monomers described by Reichert and Mathias are in contemplated embodiments used to form polymers with adamantane molecules at the core of a thermoset. The compounds disclosed by Reichert and Mathias in their studies, however, comprise only one isomer of the adamantane-based compound by design choice. Structure A shows this symmetrical para-isomer 1,3,5,7-tetrakis[4′-(phenylethynyl)phenyl]adamantane:

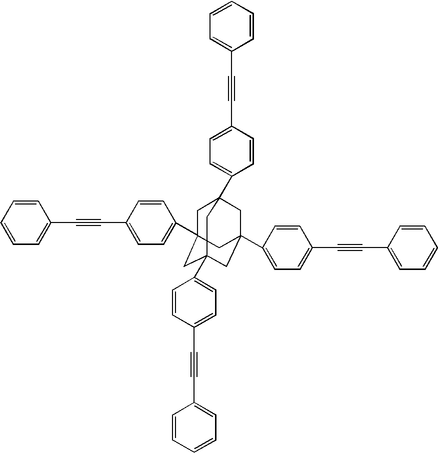

In other words, Reichert and Mathias in their individual and joint work contemplate a useful polymer comprising only one isomeric form of the target adamantane-based monomer. A significant problem exists, however, when forming and processing polymers from the single isomer form (symmetrical “all-para-” isomer) 1,3,5,7-tetrakis[4′-(phenylethynyl)phenyl]adamantane of the adamantane-based monomer. According to the Reichert dissertation (supra) and Macromolecules, vol. 27, (pp. 7015-7034) (supra), the symmetrical all-para-isomer 1,3,5,7-tetrakis[4′-(phenylethynyl)phenyl]adamantane “was found to be soluble enough in chloroform that a ¹H NMR spectrum could be obtained. However, acquisition times were found to be impractical for obtaining a solution ¹³C NMR spectrum.” Thus, the Reichert symmetrical “all-para-” isomer 1,3,5,7-tetrakis[4′-(phenylethynyl)phenyl]adamantane is insoluble in standard organic solvents and therefore, would not be useful in any application requiring solubility or solvent-based processing, such as flow coating, spin coating, or dip coating. See Comparative Example 1 below. See also U.S. Pat. No. 5,017,734.

In our commonly assigned pending patent application PCT/US01/22204 filed Oct. 17, 2001 (claiming the benefit of our commonly assigned pending patent applications U.S. Ser. No. 09/545,058 filed Apr. 7, 2000; U.S. Ser. No. 09/618,945 filed Jul. 19, 2000; U.S. Ser. No. 09/897,936 filed Jul. 5, 2001; U.S. Ser. No. 09/902,924 filed Jul. 10, 2001; and International Publication WO 01/78110 published Oct. 18, 2001), which are all included herein in their entirety, a composition comprising an isomeric thermosetting monomer or dimer mixture was discovered and disclosed, wherein the mixture comprises at least one monomer or dimer having the structure correspondingly 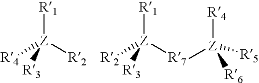
wherein Z is selected from a cage compound and a silicon atom; R′₁, R′₂, R′₃, R′₄, R′₅, and R′₆ are independently selected from an aryl, a branched aryl, and an arylene ether, and wherein at least one of the aryl, the branched aryl, and the arylene ether has an ethynyl group; and R′₇ is aryl or substituted aryl. Methods for formation of these thermosetting mixtures were also disclosed in detail. This novel isomeric thermosetting monomer or dimer mixture is useful as a dielectric material in microelectronics applications and soluble in many solvents such as cyclohexanone. These desirable properties make this isomeric thermosetting monomer or dimer mixture ideal for film formation at thicknesses of about 0.1 μm to about 1.0 μm.

In commonly assigned pending patent application 60/384403 filed May 30, 2002 and converted December 2002, which is incorporated herein in its entirety, a composition was discovered and disclosed comprising: (a) monomer of Formula A 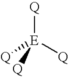
and (b) at least one oligomer or polymer of Formula B 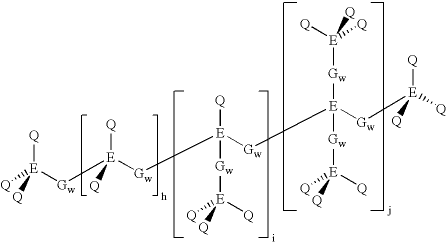
where E is a cage compound (defined below); each Q is the same or different and selected from aryl, branched aryl, and substituted aryl wherein the substituents include hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl; G_{w} is aryl or substituted aryl where substituents include halogen and alkyl; h is from 0 to 10; i is from 0 to 10; j is from 0 to 10; and w is 0 or 1. Contemplated Q groups include aryl and aryl substituted with alkenyl and alkynyl groups and more contemplated Q groups include (phenylethynyl)phenyl, phenylethynyl(phenylethynyl)phenyl, and (phenylethynyl)phenylphenyl moiety. Contemplated aryls for G_{w} include phenyl, biphenyl, and terphenyl. A more contemplated G group is phenyl.

An extremely desirable feature in the dielectric films is the tunability of film thickness from 1,000 Å to 25,000 Å. Film thicknesses for spin-on dielectrics or photoresists is controlled by spinning speed and solution viscosity. The solution viscosity is a function of matrix molecular weight, solvent, and solution concentration at a given temperature. A high molecular weight material is undesirable because film defects such as striation may occur. In our pending patent application (PCT/US01/22204 filed Oct. 17, 2001), which is commonly-owned and included herein in its entirety, our inventive mixture of the p- and m-isomers of 1,3,5,7-tetra[3′/4′(phenyethynyl)phenyl]adamantane, although an improvement over the prior art, has a very regular tetrahedral structure and thus, has limited solubility in typical organic solvents such as cyclohexanone and toluene, and also gelled and precipitated. A thick film of the mixtures previously described (greater than 6,000 Å) could not be obtained. In our pending patent application (60/384,403 filed May 30, 2002 and converted December, 2002), the mixture disclosed with an aryl linkage had reduced regional symmetry and thus, had increased solubility compared with our prior invention. Thus, a thicker film of the mixture described therein (up to 16,000 Å) could then be obtained.

Therefore, it would be beneficial to have methods of modifing the subject matter of our previous applications to improve its performance, along with descriptions of related compounds and compositions.

### SUMMARY OF THE INVENTION

Compositions and methods of forming and using those compositions are provided herein where the composition comprises at least one oligomer or polymer of Formula I 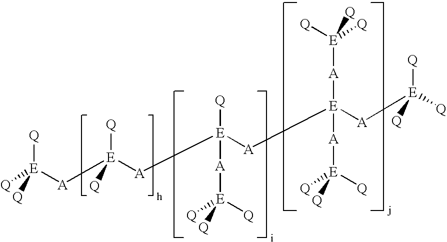
wherein E is a cage compound; each Q is the same or different and selected from aryl, branched aryl, and substituted aryl wherein the substituents include hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl; A is substituted or unsubstituted aryl with substituted or unsubstituted arylalkynyl group (substituents include hydrogen, halogen, alkyl, phenyl or substituted aryl; and aryl includes phenyl, biphenyl, naphthyl, terphenyl, anthracenyl, polyphenylene, polyphenylene ether, or substituted aryl); h is from 0 to 10; i is from 0 to 10; j is from 0 to 10; and w is 0 or 1.

### BRIEF DESCRIPTION OF THE DRAWINGS

FIG. 1 illustrates a monomer preparation method disclosed in our pending patent application PCT/US01/22204 filed Oct. 17, 2001.

FIG. 2 discusses the Reichert prior art monomer preparation method.

### DETAILED DESCRIPTION OF THE INVENTION

Methods of modifing the subject matter of our previous applications to improve its performance have in fact been discovered and are described herein according to the goals previously stated, along with descriptions of related compounds and compositions.

A composition comprising at least one oligomer or polymer of Formula I 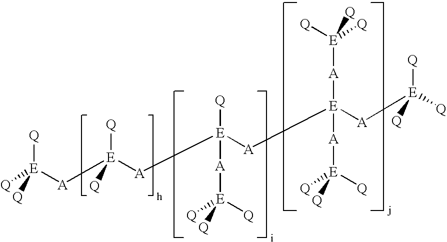
wherein E is a cage compound; each Q is the same or different and selected from aryl, branched aryl, and substituted aryl wherein the substituents include hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl; A is substituted or unsubstituted aryl with substituted or unsubstituted arylalkynyl group (substituents include hydrogen, halogen, alkyl, phenyl or substituted aryl; and aryl includes phenyl, biphenyl, naphthyl, terphenyl, anthracenyl, polyphenylene, polyphenylene ether, or substituted aryl); h is from 0 to 10; i is from 0 to l0; j is from 0 to 10; and w is 0 or 1.

The phrases “cage structure”, “cage molecule”, and “cage compound” are intended to be used interchangeably and refer to a molecule having at least 10 atoms arranged such that at least one bridge covalently connects two or more atoms of a ring system. In other words, a cage structure, cage molecule or cage compound comprises a plurality of rings formed by covalently bound atoms, wherein the structure, molecule or compound defines a volume, such that a point located with the volume can not leave the volume without passing through the ring. The bridge and/or the ring system may comprise one or more heteroatoms, and may be aromatic, partially saturated, or unsaturated. Further contemplated cage structures include fullerenes, and crown ethers having at least one bridge. For example, an adamantane or diamantane is considered a cage structure, while a naphthalene or an aromatic spirocompound are not considered a cage structure under the scope of this definition, because a naphthalene or an aromatic spirocompound do not have one, or more than one bridge.

Contemplated cage compounds need not necessarily be limited to being comprised solely of carbon atoms, but may also include heteroatoms such as N, S, O, P, etc. Heteroatoms may advantageously introduce non-tetragonal bond angle configurations. With respect to substituents and derivatizations of contemplated cage compounds, it should be recognized that many substituents and derivatizations are appropriate. For example, where the cage compounds are relatively hydrophobic, hydrophilic substituents may be introduced to increase solubility in hydrophilic solvents, or vice versa. Alternatively, in cases where polarity is desired, polar side groups may be added to the cage compound. It is further contemplated that appropriate substituents may also include thermolabile groups, nucleophilic and electrophilic groups. It should also be appreciated that functional groups may be employed in the cage compound (e.g., to facilitate crosslinking reactions, derivatization reactions, etc.) Where the cage compounds are derivatized, it is especially contemplated that derivatizations include halogenation of the cage compound, and a particularly preferred halogen is fluorine.

Cage molecules or compounds, as described in detail herein, can also be groups that are attached to a polymer backbone, and therefore, can form nanoporous materials where the cage compound forms one type of void (intramolecular) and where the crosslinking of at least one part of the backbone with itself or another backbone can form another type of void (intermolecular). Additional cage molecules, cage compounds and variations of these molecules and compounds are described in detail in PCT/US01/32569 filed on Oct. 18, 2001, which is herein incorporated by reference in its entirety.

In contrast to our two prior inventions discussed above, the subject matter described herein, by having reduced regional symmetry is more soluble in typical organic solvents and thus, provides greater film thicknesses (up to 19,000 Å). In addition, the present compositions advantageously provide flexibility and low melt viscosities.

In contemplated embodiments, the composition comprises at least one oligomer or polymer of adamantane monomer of Formula II: 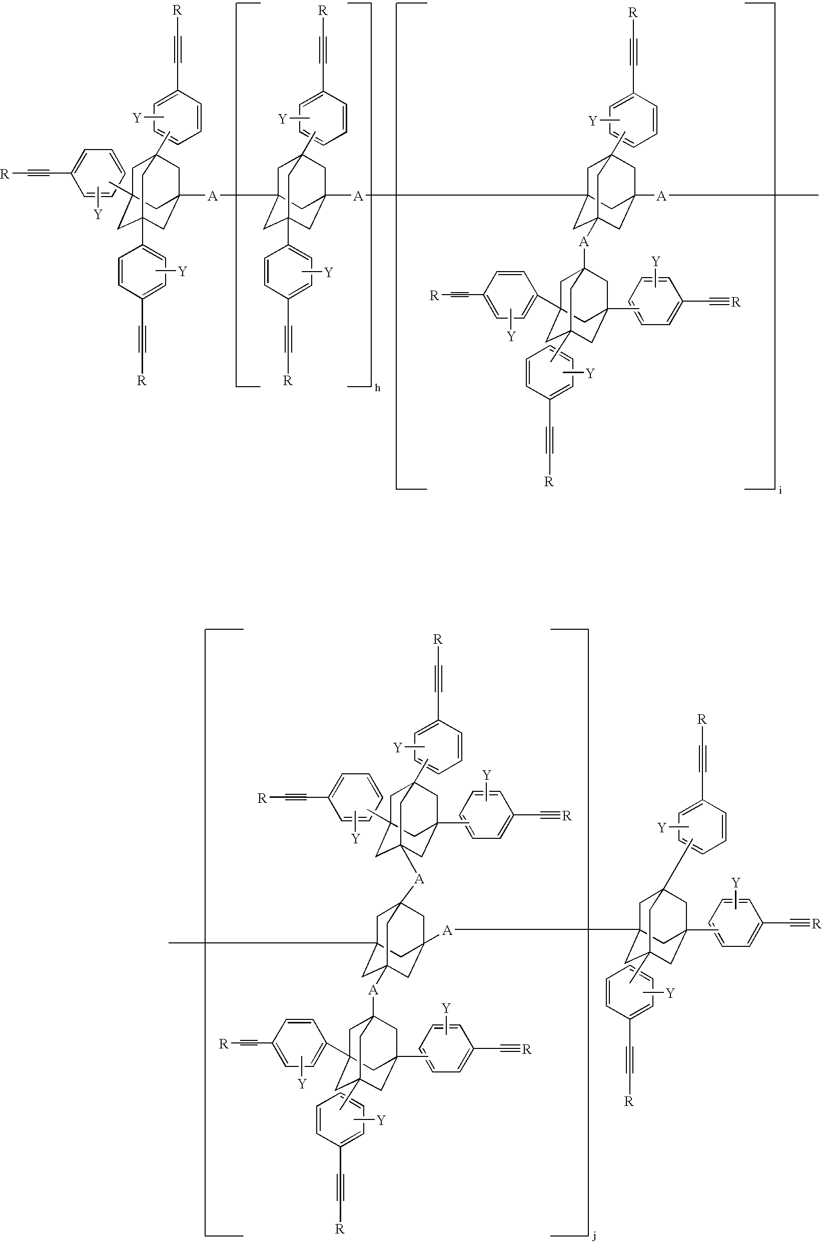
or at least one oligomer or polymer of diamantane monomer of Formula III: 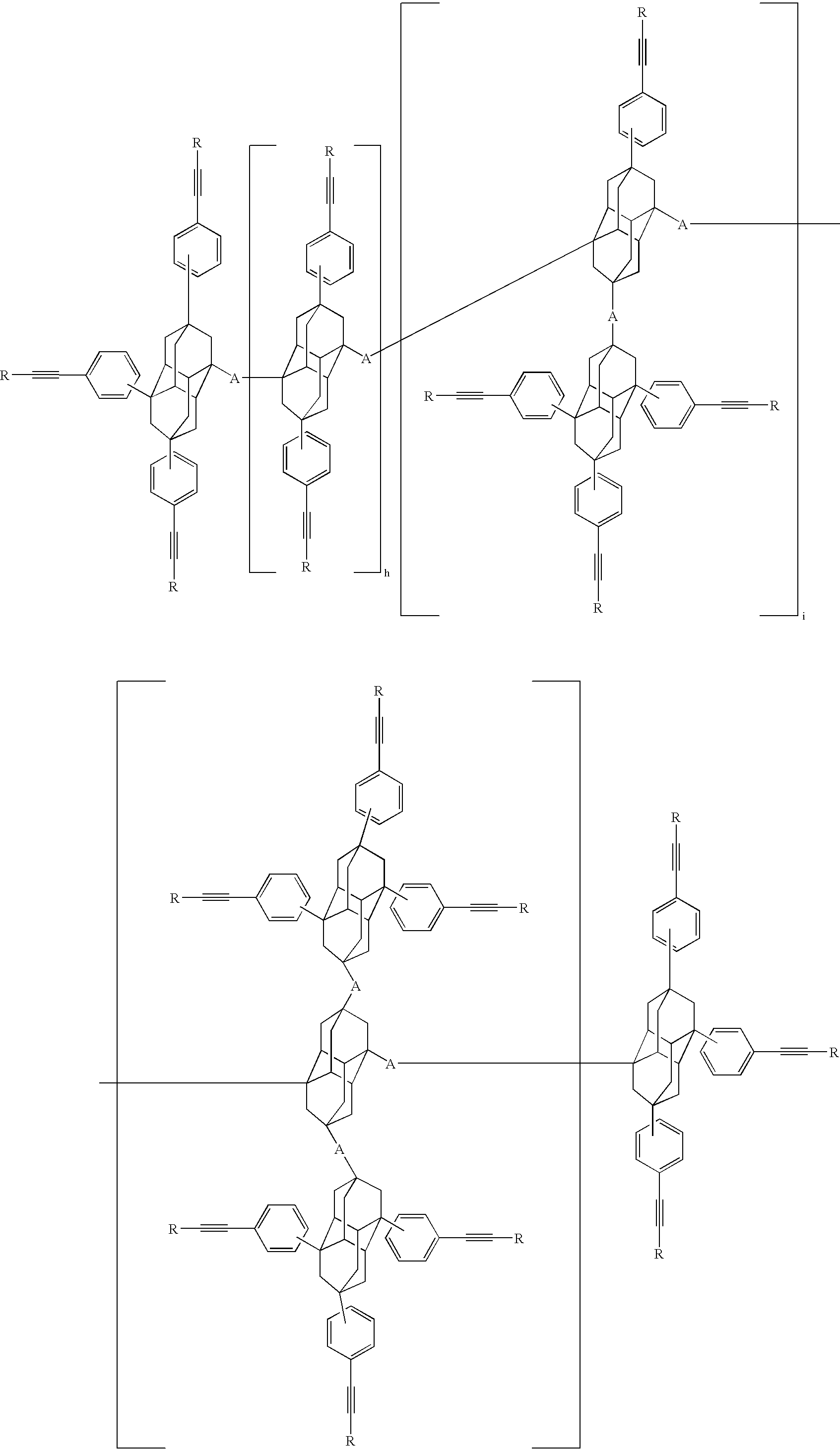
wherein h is from 0 to 10; i is from 0 to 10; j is from 0 to 0; each R in Formulae II and III is the same or different and selected from hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl; and each A in Formulae II and III is the same or different and comprises substituted or unsubstituted aryl with substituted or unsubstituted arylalkynyl groups. Substituents include hydrogen, halogen, alkyl, phenyl or substituted aryl; and aryl includes phenyl, biphenyl, naphthyl, terphenyl, anthracenyl, polyphenylene, polyphenylene ether, or substituted aryl.

In other contemplated embodiments, A is Formula IV 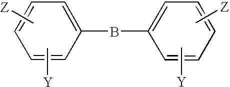
wherein B comprises the formula:
(Ar)ₙ—[Ar₂—(C≡C—)ₓ]_{y}
where n is 0, 1, 2, or 3; x is 1, 2, 3, 4, or 5; y is 1, 2, 3, 4, or 5 and xy≧2.Ar₁ and Ar₂ are same or different and are substituted or unsubstituted aryls. Substituents include hydrogen, halogen, alkyl, phenyl or substituted aryl; and aryl includes phenyl, biphenyl, naphthyl, terphenyl, anthracenyl, polyphenylene, polyphenylene ether, or substituted aryl; Y is the same Y as above; and Z is hydrogen, phenylethynyl, or the same as B above.

In yet other contemplated embodiments, B is selected from the following:

| NAME | STRUCTURE |
|---|---|
| m-diethynyl benzene (hereinafter “m-DEB”) | 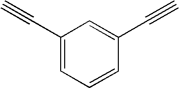 |
| p-diethynyl benzene (hereinafter “p-DEB”) | 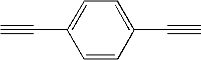 |
| Diethynyl naphthalene (hereinafter “DEN”) | 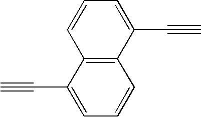 |
| Diethynyl biphenyl (hereinafter “BEBP”) | 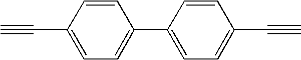 |
| | 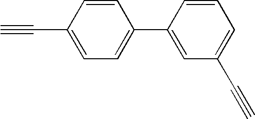 |
| 9,10-diethynyl anthracene | 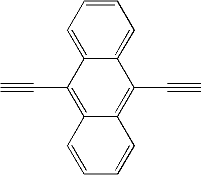 |
| 1,5-diethynyl anthracene | 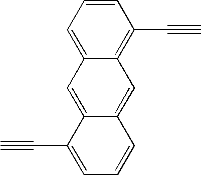 |

Formulae II and III represent random or irregular structures in that any one of the units h, i, and j may or may not repeat numerous times before another unit is present. Thus, the sequence of units in Formulae II and III above is random or irregular.

In some contemplated embodiments, the composition comprises at least one adamantane oligomer or polymer of Formula II above where h is 0 or 1, i is 0, and j is 0. This adamantane structure is shown as Formula V below where R, Y, and A are as defined above.

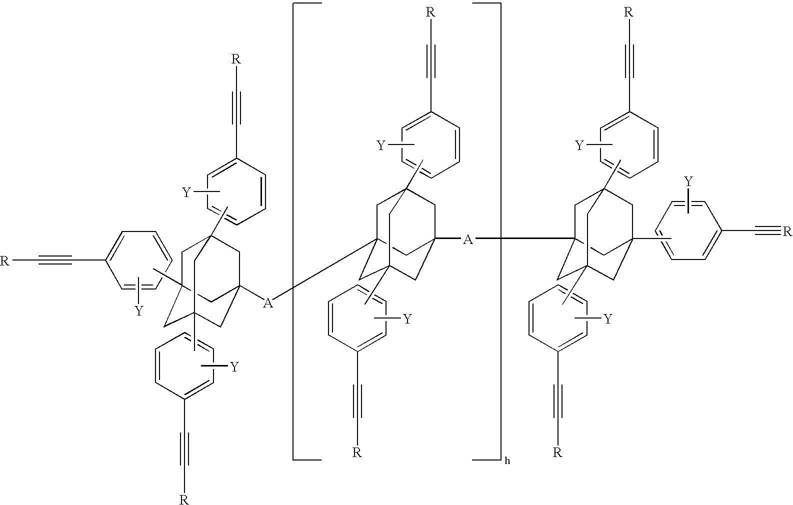

In other contemplated embodiments, the composition comprises at least one diamantane oligomer or polymer of Formula II[ above where h is 0 or 1, i is 0, and j is 0. This diamantane structure is shown as Formula VI below where R, Y, and A are as defined above.

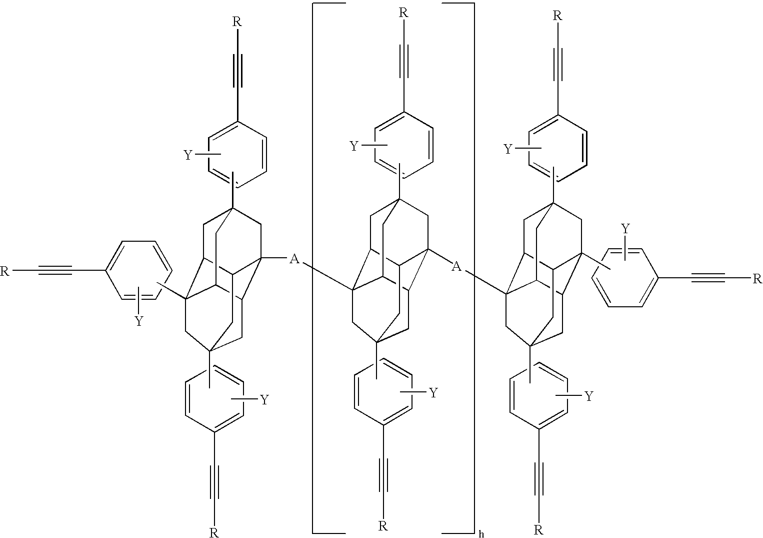

In yet other contemplated embodiments, the composition comprises at least one adamantane oligomer or polymer of Formula V above where h is 0. This adamantane dimer is shown as Formula VII below where R, Y, and A are as defined above.

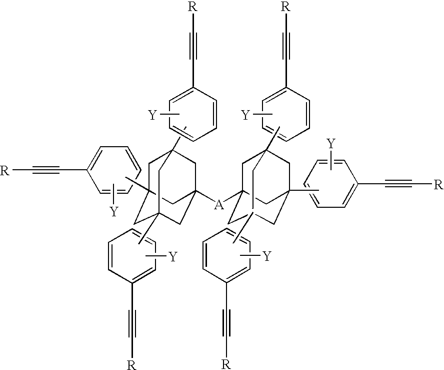

In additional contemplated embodiments, the composition comprises at least one diamantane oligomer or polymer of Formula VI above where h is 0. This diamantane dimer is shown as Formula VIII below where R, Y, and A are as defined above.

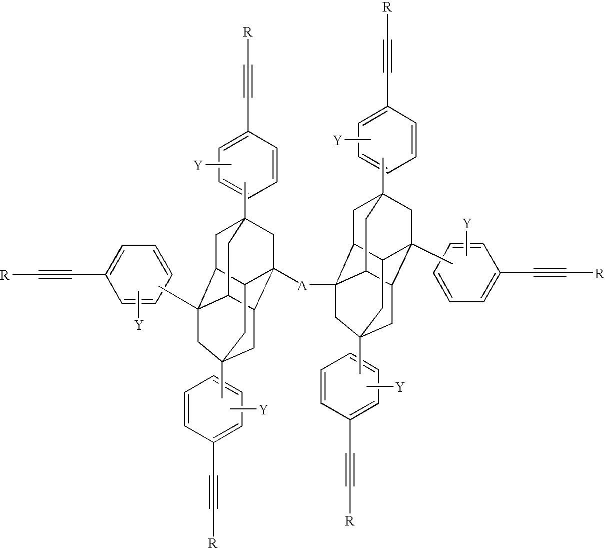

In some embodiments, the composition comprises at least one adamantane oligomer or polymer of Formula V above where h is 1. This adamantane trimer is as shown in Formula IX below where R, Y, and A are as defined above.

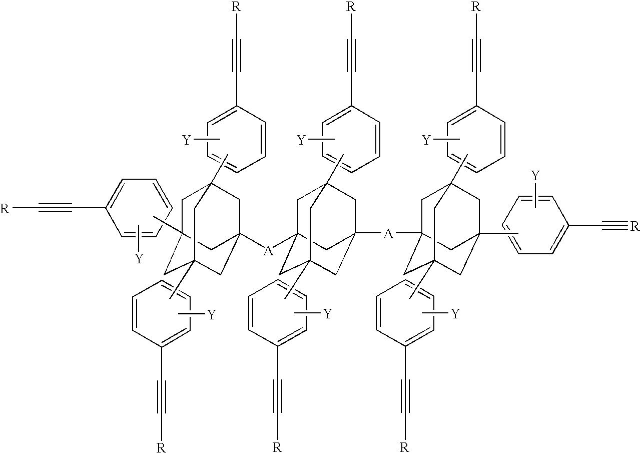

In other contemplated embodiments, the composition comprises at least one diamantane oligomer or polymer of Formula VI above where h is 1. This diamantane trimer is as shown in Formula X below where R, Y, and A are as defined above.

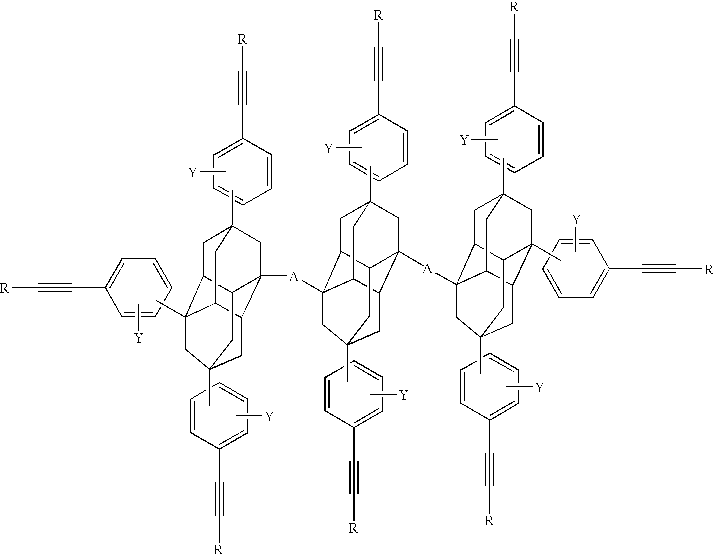

In yet other contemplated embodiments, the composition comprises at least one adamantane oligomer or polymer of Formula II above where h is 2, i is 0, and j is 0 (linear oligomer or polymer) and h is 0, i is 1, and j is 0 (branched oligomer or polymer). Thus, this composition comprises an adamantane linear tetramer as shown in Formula XI below where R, Y, and A are as defined above; 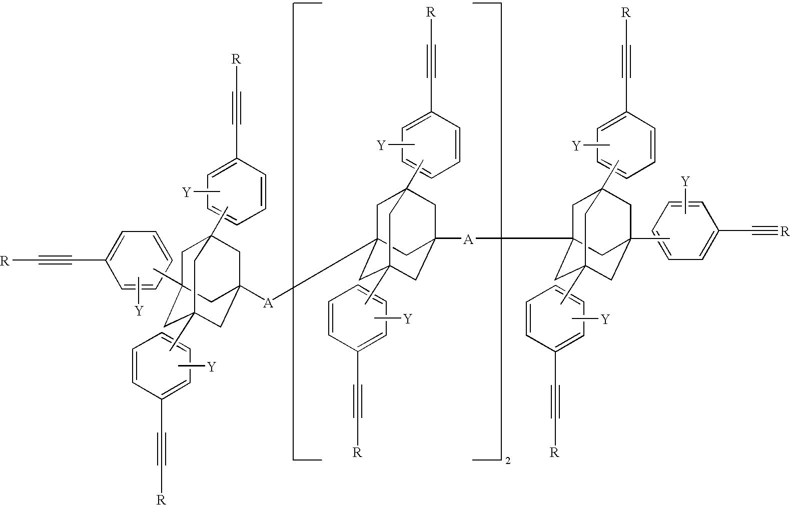
and adamantane branched tetramer as shown Formula XII below where R, Y, and A are as defined above.

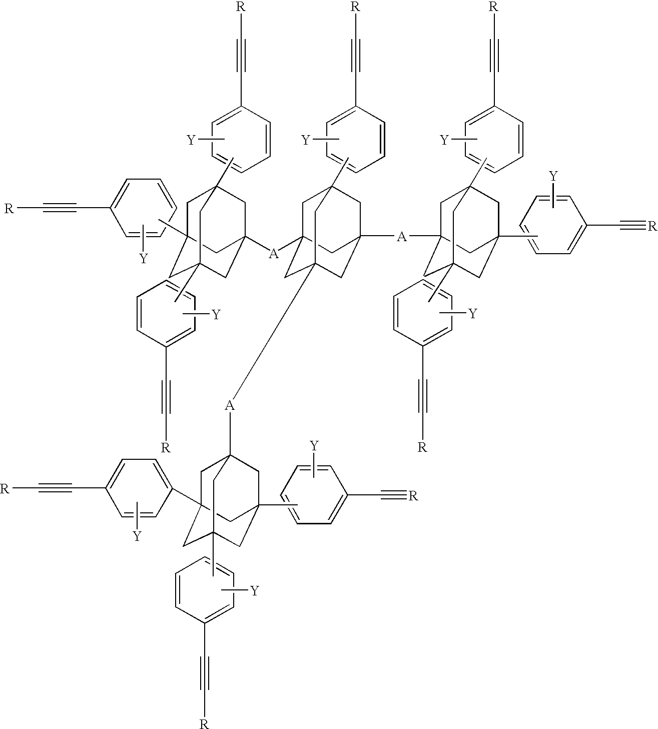

In several contemplated embodiments, the composition comprises at least one diamantane oligomer or polymer of Formula III above where h is 2, i is 0, and j is 0 (linear oligomer or polymer) and h is 0, i is 1, and j is 0 (branched oligomer or polymer). Thus, the present composition comprises diamantane linear tetramer as shown in Formula XIII below where R, Y, and A are as defined above 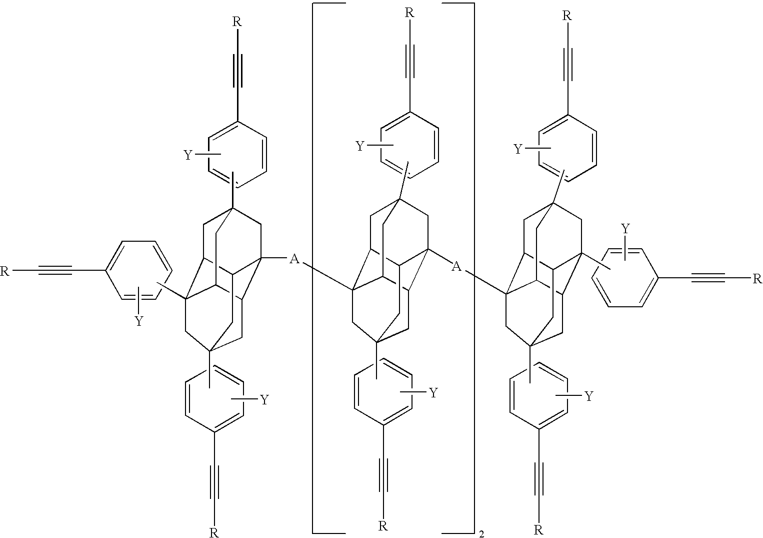
and diamantane branched tetramer as shown Formula XIV below where R, Y, and A are as defined above.

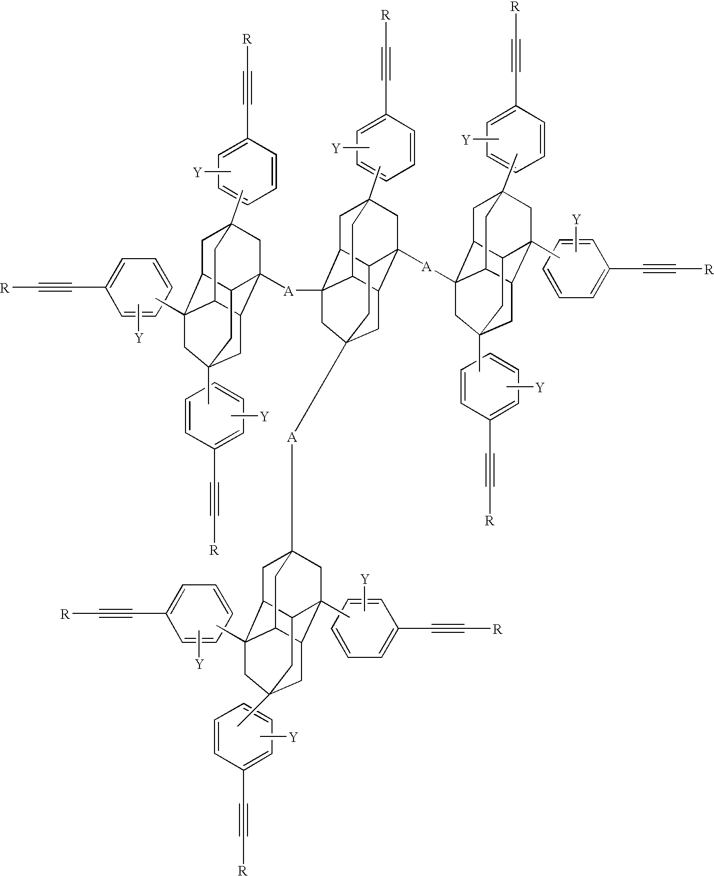

In some embodiments, the composition comprises adamantane dimer of Formula VII above and adamantane trimer of Formula IX above. In contemplated embodiments, the present composition comprises diamantane dimer of Formula VII above and diamantane trimer of Formula X above.

In other embodiments, the composition comprises adamantane dimer of Formula VII above and at least one adamantane oligomer or polymer of Formula II above where at least one of h, i, and j is at least 1. In yet other contemplated embodiments, the composition comprises diamantane dimer of Formula VIII above and at least one diamantane oligomer or polymer of Formula III above where at least one of h, i, and j is at least 1.

Thermosetting Component:

The phrase “bridgehead carbon” as used herein refers to any cage structure carbon bound to three other carbons. Thus, for example, adamantane has four bridgehead carbons while diamantane has eight bridgehead carbons.

The phrase “low dielectric constant polymer” and/or “low dielectric constant material” are intended to be used interchangeably and as used herein refer to an organic, organometallic, or inorganic polymer with a dielectric constant of approximately 3.0 or lower.

The low dielectric material is typically manufactured in the form of a thin layer having a thickness from 100 to 25,000 Angstroms but also may be used as thick films, blocks, cylinders, spheres etc. In some embodiments, at least one layer comprises a thickness of up to about 25,000 Angstroms. In other embodiments, the at least one layer comprises a thickness of up to about 16,000 Angstroms. In yet other embodiments, the at least one layer comprises a thickness of up to about 10,000 Angstroms. In additional embodiments, the at least one layer comprises a thickness of up to about 5,000 Angstroms. In yet other embodiments, the at least one layer comprises a thickness of up to about 1,000 Angstroms.

The term “layer” as used herein includes a film and/or coating as applied to a surface and/or substrate. Substrates and surfaces contemplated herein may comprise any desirable substantially solid material. Particularly desirable substrate layers would comprise films, glass, ceramic, plastic, metal or coated metal, or composite material. In some embodiments, the substrate comprises a silicon or germanium arsenide die or wafer surface, a packaging surface such as found in a copper, silver, nickel or gold plated leadframe, a copper surface such as found in a circuit board or package interconnect trace, a via-wall or stiffener interface (“copper” includes considerations of bare copper and its oxides), a polymer-based packaging or board interface such as found in a polyimide-based flex package, lead or other metal alloy solder ball surface, glass and polymers such as polyimide. In other embodiments, the substrate comprises a material common in the packaging and circuit board industries such as silicon, copper, glass, and another polymer.

Generally, h, i, and j are whole numbers from 0 to 10, in contemplated embodiments 0 to 5, and in more contemplated embodiments 0 to 2. The simplest adamantane oligomer is thus the dimer (h is 0, i is 0, and j is 0 in Formula II above) as shown in Formula VII above, in which two adamantane frameworks are linked via an unsubstituted or substituted aryl unit. The simplest diamantane oligomer is thus the dimer (h is 0, i is 0, and j is 0 in Formula III above) as shown in Formula VIII above, in which two diamantane frameworks are linked via an unsubstituted or substituted aryl unit.

The individual radicals R of the substituted ethynyl radical on the phenyl ring attached to the adamantane or diamantane ring of the type RC≡C— are in each case the same or different in Formulae II, III, IV, V, VI, VII, VIII, IX, X, XI, XII, XIII, and XIV. R is selected from hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl. Each R may be unbranched or branched and unsubstituted or substituted and said substituents may be unbranched or branched. It is contemplated that the radicals alkyl, alkenyl, alkynyl, alkoxyl, hydroxyalkyl, hydroxyalkenyl, and hydroxyalkynyl contain from about 2 to about 10 carbon atoms and the radicals aryl, aryl ether, and hydroxyaryl contain from about 6 to about 18 carbon atoms. If R stands for aryl, R is in contemplated embodiments phenyl. In contemplated embodiments, at least two of the RC_=C groups on the phenyl groups are two different isomers. Examples of at least two different isomers include meta-, para-, and ortho-isomers. In contemplated embodiments, the at least two different isomers are meta- and para-isomers.

Each Y of the phenyl rings in the Formulae III, IV, V, VI, VII, VII, IX, X, XI, XII, XIII, and XIV is in each case the same or different and selected from hydrogen, alkyl, aryl, substituted aryl, or halogen. When Y is aryl, examples of aryl groups include phenyl or biphenyl. Y is selected from in contemplated embodiments hydrogen, phenyl, and biphenyl and more in contemplated embodiments hydrogen.

The term “alkyl” is used herein to mean a branched or a straight-chain saturated hydrocarbon group or substituent of 1 to 24 carbon atoms, such as methyl, ethyl, n-propyl, isopropyl, n-butyl, isobutyl, t-butyl, octyl, decyl, tetradecyl, hexadecyl, eicosyl, tetracosyl and the like. In some embodiments, contemplated alkyl groups contain 1 to 12 carbon atoms. The term “cyclic alkyl” means an alkyl compound whose structure is characterized by one or more closed rings. The cyclic alkyl may be mono-, bi-, tri- or polycyclic depending on the number of rings present in the compound. The term “aryl” is used herein to mean a monocyclic aromatic species of 5 to 7 carbon atoms or a compound that is built with monocyclic aromatic species of 5 to 7 carbon atoms and is typically phenyl, naphthalyl, phenanthryl, anthracyl etc. Optionally, these groups are substituted with one to four, more preferably one to two alkyl, alkoxy, hydroxy, and/or nitro substituents.

The term “alkenyl” is used herein to mean a branched or a straight-chain hydrocarbon chain containing from 2 to 24 carbon atoms and at least one double bond. Preferred alkenyl groups herein contain 1 to 12 carbon atoms. The term “arylalkylene” is used herein to mean moieties containing both alkylene and monocyclic aryl species, typically containing less than about 12 carbon atoms in the alkylene portion, and wherein the aryl substituent is bonded to the structure of interest through an alkylene linking group. Exemplary arylalkylene groups have the structure —(CH₂)ⱼ—Ar wherein “j” is an integer in the range of 1 to 6 and wherein “Ar” is an aryl species.

In addition to the branched adamantane structure of Formula XII above, it should be understood that Formula II above when h is 0, i is 0, and j is 1 represents further branching as shown in Formula XV below. It should be understood that branching may occur beyond that of the Formula XV structure because further branching of the pending adamantane units of the Formula XV structure may also occur.

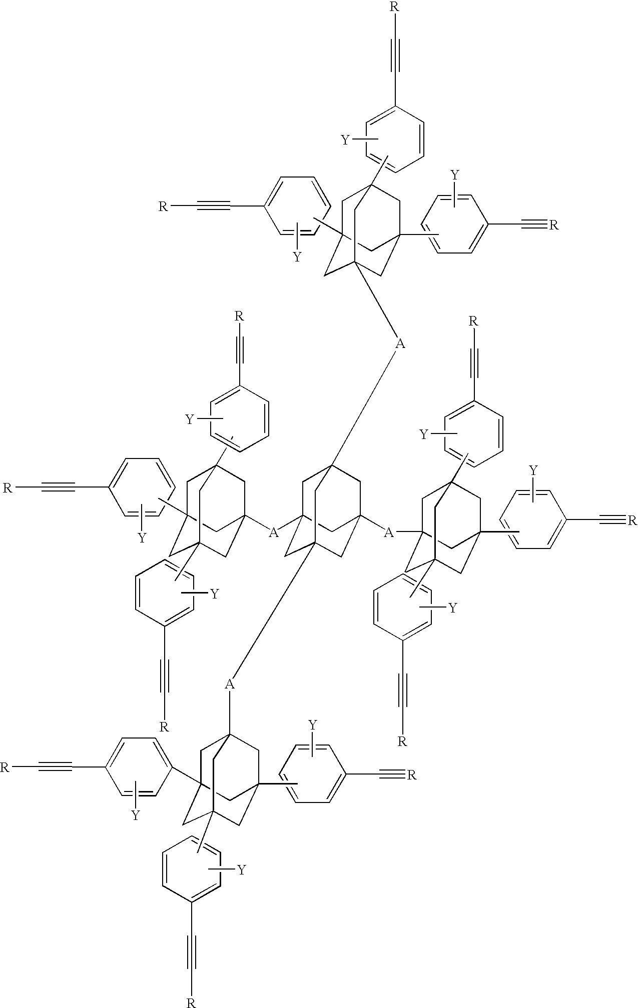

In addition to the branched diamantane structure of Formula XIV above, it should be understood that Formula III above when h is 0, i is 0, and j is 1 represents further branching as shown in Formula XVI below. It should be understood that branching may occur beyond that of the Formula XVI structure because further branching of the pending diamantane units of the Formula XVI structure may also occur. 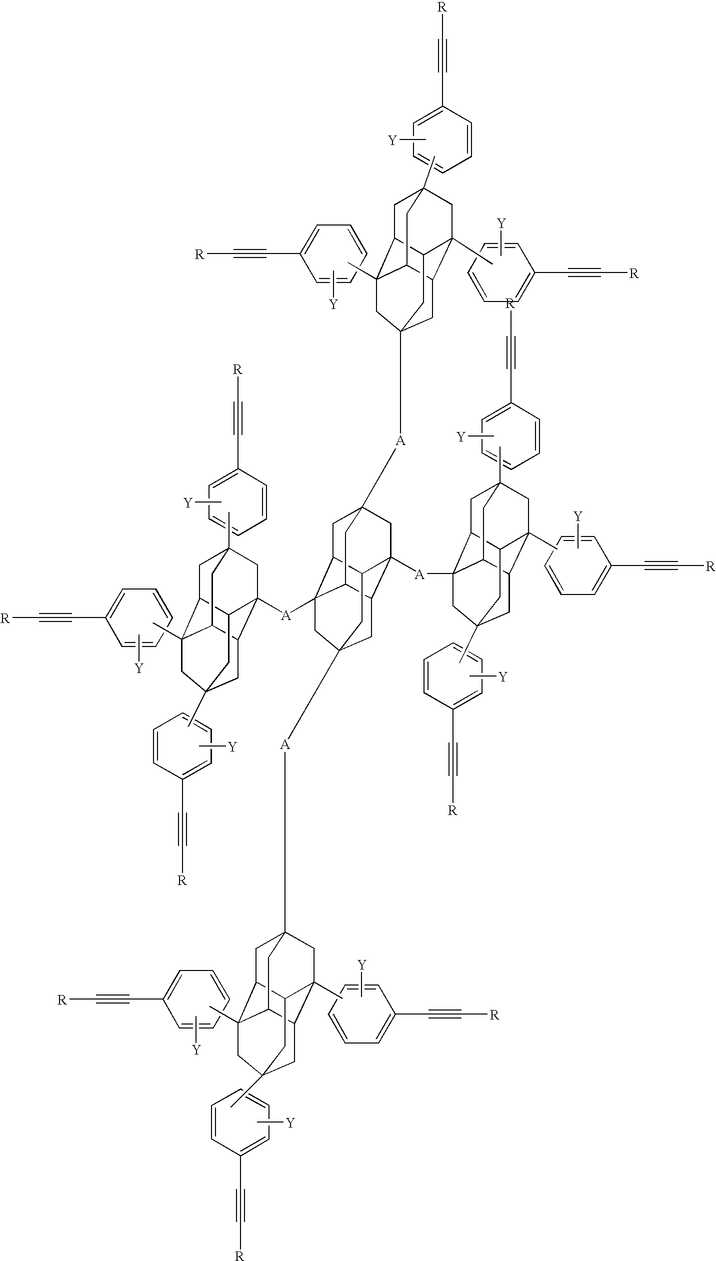
Adhesion Promoter:

In contemplated embodiments, an adhesion promoter is added to the composition described herein. The adhesion promoter may be a comonomer reacted with the present composition or an additive to the present composition.

The phrase “adhesion promoter” as used herein means any component that when used with the thermally degradable polymer, improves the adhesion thereof to substrates compared with thermally degradable polymers. Preferably the at least one adhesion promoter is used with the thermally degradable polymer. The adhesion promoter may be a co-monomer reacted with the thermally degradable polymer precursor or an additive to the thermally degradable polymer precursor. Examples of useful adhesion promoters are disclosed in commonly assigned pending U.S. application Ser. No. 158,513 filed May 30, 2002 incorporated herein in its entirety.

Adhesion promoters contemplated herein may comprise compounds having at least bifunctionality wherein the bifunctionality may be the same or different and at least one of said first functionality and said second functionality is selected from the group consisting of Si-containing groups; N-containing groups; C bonded to O-containing groups; hydroxyl groups; and C double bonded to C-containing groups. The phrase “compound having at least bifunctionality” as used herein means any compound having at least two functional groups capable of interacting or reacting, or forming bonds as follows. The functional groups may react in numerous ways including addition reactions, nucleophilic and electrophilic substitutions or eliminations, radical reactions, etc. Further alternative reactions may also include the formation of non-covalent bonds, such as Van der Waals, electrostatic bonds, ionic bonds, and hydrogen bonds.

In some embodiments of the at least one adhesion promoter, preferably at least one of the first functionality and the second functionality is selected from Si-containing groups; N-containing groups; C bonded to O-containing groups; hydroxyl groups; and C double bonded to C-containing groups. Preferably, the Si-containing groups are selected from Si—H, Si—O, and Si—N; the N-containing groups are selected from such as C—NH₂ or other secondary and tertiary amines, imines, amides, and imides; the C bonded to O-containing groups are selected from ═CO, carbonyl groups such as ketones and aldehydes, esters, —COOH, alkoxyls having 1 to 5 carbon atoms, ethers, glycidyl ethers; and epoxies; the hydroxyl group is phenol; and the C double bonded to C-containing groups are selected from allyl and vinyl groups. For semiconductor applications, the more preferred functional groups include the Si-containing groups; C bonded to O-containing groups; hydroxyl groups; and vinyl groups.

Contemplated adhesion promoters may also comprise an organic resin-based material that further comprises phenolic-containing resins, novolac resins, such as CRJ-406 or HRJ-11040 (both from Schenectady International, Inc.), organic acrylate and/or a styrene resins. Other adhesion promoters may comprise polydimethylsiloxane materials, ethoxy or hydroxy-containing silane monomers, vinyl-containing silane monomers, acrylated silane monomers, or silyl hydrides.

An example of a contemplated adhesion promoter having Si-containing groups is silanes of the Formula I: (R₁₄)ₖ(R₁₅)ₗSi(R₁₆)ₘ(R₁₇)ₙ wherein R₁₄, R₁₅, R₁₆, and R₁₇ each independently represents hydrogen, hydroxyl, unsaturated or saturated alkyl, substituted or unsubstituted alkyl where the substituent is amino or epoxy, saturated or unsaturated alkoxyl, unsaturated or saturated carboxylic acid radical, or aryl; at least two of R₁₄, R,₅, R₁₆, and R₁₇ represent hydrogen, hydroxyl, saturated or unsaturated alkoxyl, unsaturated alkyl, or unsaturated carboxylic acid radical; and k+l+m+n≦4. Examples include vinylsilanes such as H₂C═CHSi(CH₃)₂H and H₂C═CHSi(R₁₈)₃ where R₁₈ is CH₃O, C₂H₅O, AcO, H₂C═CH, or H₂C═C(CH₃)O—, or vinylphenylmethylsilane; allylsilanes of the formula H₂C═CHCH₂—Si(OC₂H₅)₃ and H₂C═CHCH₂—Si(H)(OCH₃)₂; glycidoxypropylsilanes such as (3-glycidoxypropyl)methyldiethoxysilane and (3-glycidoxypropyl)trimethoxysilane; methacryloxypropylsilanes of the formula H₂C═(CH₃)COO(CH₂)₃—Si(OR₁₉)₃ where R₁₉ is an alkyl, preferably methyl or ethyl; aminopropylsilane derivatives including H₂N(CH₂)₃Si(OCH₂CH₃)₃, H₂N(CH₂)₃Si(OH)₃, or H₂N(CH₂)₃OC(CH₃)₂CH═CHSi(OCH₃)₃. The aforementioned silanes are commercially available from Gelest.

An example of a preferred adhesion promoter having C bonded to O-containing groups is glycidyl ethers including but not limited to 1,1,1-tris-(hydroxyphenyl)ethane tri-glycidyl ether which is commercially available from TriQuest.

An example of a preferred adhesion promoter having C bonded to O-containing groups is esters of unsaturated carboxylic acids containing at least one carboxylic acid group. Examples include trifunctional methacrylate ester, trifunctional acrylate ester, trimethylolpropane triacrylate, dipentaerytbritol pentaacrylate, and glycidyl methacrylate. The foregoing are all commercially available from Sartomer.

An example of a preferred adhesion promoter having vinyl groups is vinyl cyclic pyridine oligomers or polymers wherein the cyclic group is pyridine, aromatic, or heteroaromatic. Useful examples include but not limited to 2-vinylpyridine and 4-vinylpyridine, commercially available from Reilly; vinyl aromatics; and vinyl heteroaromatics including but not limited to vinyl quinoline, vinyl carbazole, vinyl imidazole, and vinyl oxazole.

An example of a preferred adhesion promoter having Si-containing groups is the polycarbosilane disclosed in commonly assigned copending allowed U.S. patent application Ser. No. 09/471,299 filed Dec. 23, 1999 incorporated herein by reference in its entirety. The polycarbosilane is that shown in Formula II: 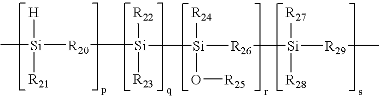
in which R₂₀, R₂₆, and R₂₉ each independently represents substituted or unsubstituted alkylene, cycloalkylene, vinylene, allylene, or arylene; R₂₁, R₂₂, R₂₃, R₂₄, R₂₇, and R₂₈ each independently represents hydrogen atom or organo group comprising alkyl, alkylene, vinyl, cycloalkyl, allyl, or aryl and may be linear or branched; R₂₅ represents organosilicon, silanyl, siloxyl, or organo group; and p, q, r, and s satisfy the conditions of [4≦p+q+r+s≦100,000], and q and r and s may collectively or independently be zero. The organo groups may contain up to 18 carbon atoms but generally contain from about 1 to about 10 carbon atoms. Useful alkyl groups include —CH₂— and —(CH₂)ₜ— where t>1.

Contemplated polycarbosilanes include dihydridopolycarbosilanes in which R₂₀ is a substituted or unsubstituted alkylene or phenyl, R₂₁ group is a hydrogen atom and there are no appendent radicals in the polycarbosilane chain; that is, q, r, and s are all zero. Another preferred group of polycarbosilanes are those in which the R₂₁, R₂₂, R₂₃, R₂₄, R₂₅, and R₂₈ groups of Formula II are substituted or unsubstituted alkenyl groups having from 2 to 10 carbon atoms. The alkenyl group may be ethenyl, propenyl, allyl, butenyl or any other unsaturated organic backbone radical having up to 10 carbon atoms. The alkenyl group may be dienyl in nature and includes unsaturated alkenyl radicals appended or substituted on an otherwise alkyl or unsaturated organic polymer backbone. Examples of these preferred polycarbosilanes include dihydrido or alkenyl substituted polycarbosilans such as polydihydridocarbosilane, polyallylhydrididocarbosilane and random copolymers of polydihydridocarbosilane and polyallylhydridocarbosilane.

In the more preferred polycarbosilanes, the R₂₁ group of Formula II is a hydrogen atom and R₂₁ is methylene and the appendent radicals q, r, and s are zero. Other preferred polycarbosilane compounds of the invention are polycarbosilanes of Formula II in which R₂₁ and R₂₇ are hydrogen, R₂₀ and R₂₉ are methylene, and R₂₈ is an alkenyl, and appendent radicals q and r are zero. The polycarbosilanes may be prepared from well known prior art processes or provided by manufacturers of polycarbosilane compositions. In the most preferred polycarbosilanes, the R₂₁ group of Formula II is a hydrogen atom; R₂₄ is —CH₂—; q, r, and s are zero and p is from 5 to 25. These most preferred polycarbosilanes may be obtained from Starfire Systems, Inc. Specific examples of these most preferred polycarbosilanes follow:

| | Weight Average | | Peak Molecular |
|---|---|---|---|
| | Molecular Weight | | Weight |
| Polycarbosilane | (Mw) | Polydispersity | (Mp) |
| 1 | 400-1,400 | 2-2.5 | 330-500 |
| 2 | 330 | 1.14 | 320 |
| 3 (with 10% | 10,000-14,000 | 10.4-16 | 1160 |
| allyl groups) | | | |
| 4 (with 75% | 2,400 | 3.7 | 410 |
| allyl groups) | | | |

As can be observed in Formula II, the polycarbosilanes utilized may contain oxidized radicals in the form of siloxyl groups when r>0. Accordingly, R₂₅ represents organosilicon, silanyl, siloxyl, or organo group when r>0. It is to be appreciated that the oxidized versions of the polycarbosilanes (r>0) operate very effectively in, and are well within the purview of the 25 present invention. As is equally apparent, r can be zero independently of p, q, and s the only conditions being that the radicals p, q, r, and s of the Formula II polycarbosilanes must satisfy the conditions of [4<p+q+r+s<100,000], and q and r can collectively or independently be zero.

The polycarbosilane may be produced from starting materials that are presently commercially available from many manufacturers and by using conventional polymerization processes. As an example of synthesis of the polycarbosilanes, the starting materials may be produced from common organo silane compounds or from polysilane as a starting material by heating an admixture of polysilane with polyborosiloxane in an inert atmosphere to thereby produce the corresponding polymer or by heating an admixture of polysilane with a low molecular weight carbosilane in an inert atmosphere to thereby produce the corresponding polymer or by heating an admixture of polysilane with a low molecular carbosilane in an inert atmosphere and in the presence of a catalyst such as polyborodiphenylsiloxane to thereby produce the corresponding polymer. Polycarbosilanes may also be synthesized by Grignard Reaction reported in U.S. Pat. No. 5,153,295 hereby incorporated by reference in its entirety.

An example of a preferred adhesion promoter having hydroxyl groups is phenol-formaldehyde resins or oligomers of the Formula III: —[R₃₀C₆H₂(OH)(R₃₁)ᵤ— where R₃₀ is substituted or unsubstituted alkylene, cycloalkylene, vinyl, allyl, or aryl; R₃₁ is alkyl, alkylene, vinylene, cycloalkylene, allylene, or aryl; and u=3-100. Examples of useful alkyl groups include —CH₂— and —(CH₂)ᵥ— where v>1. A particularly useful phenol-formaldehyde resin oligomer has a molecular weight of 1500 and is commercially available from Schenectady International Inc.

The present adhesion promoter is added in small, effective amounts in contemplated embodiments from about 1% to about 10% and more in contemplated embodiments from about 2% to about 7% based on the weight of the present composition.

Porogen:

In contemplated embodiments, porogen is added to the present composition. The pores or voids may be formed as a result of structural rearrangement or loss of material such that a pore or void or increase in free volume is left behind. The pores or voids in the material, coating and/or film create additional surface area in the coating or film which ultimately increases the etch selectivity and/or stripping selectivity of the material, coating and/or film. The porosity of the fill material, generally, is about the same as the porosity of the dielectric material, and in both instances, the porosity is greater than the porosity of the photoresist material. These dry etch selectivities are sometimes necessary to maintain proper transfer of the critical dimension from photoresist patterning through etch. The porogen's molecular weight can also be used to determine if the porogen is compatible with the absorbing composition and/or coating compound's matrix in the material. This compatibility quotient is related to the solubility parameters of the absorbing composition and/or coating compound's matrix. In an ideal case the porogen should match the solubility parameter of the matrix coating formulation before bake, so that when formulation molecular weights are known, appropriate molecular weights of the porogen can be determined by matching the solubility parameters with the matrix. Solubility parameters may be determined experimentally by relationships to the film defects, dielectric constant, wet etching tests, defect inspection through microscopes or scanning electron microscopy, or by calculation using group contribution methods or by molecular models of cohesive energy. (review ref Physical Properties of Polymers Handbook, Chapter 16 “Solubility Parmaters” Y. Du, Y. Xue, H. L. Frisch pp 227-239; James E. Mark Ed., 1996, American Institute of Physics, Woodbury, N.Y.).

As used herein, the term “pore” includes voids and cells in a material and any other term meaning space occupied by gas in the material. The term “pore” may also include a differential in material density wherein the free volume has been increased (“porous nature” has been introduced). Appropriate gases include relatively pure gases and mixtures thereof. Air, which is predominantly a mixture of N₂ and O₂ is commonly distributed in the pores, but pure gases such as nitrogen, helium, argon, CO₂ or CO are also contemplated. Pores are typically spherical but may alternatively or additionally include tubular, lamellar, discoidal, voids having other shapes, or a combination of the preceding shapes and may be open or closed. The term “porogen” as used herein may have a variety of mechanisms available to form the pore but in general is a material which upon removal leaves behind either a “pore” or a “void” or a material that can rearrange to create a “pore” or “void”. In one embodiment, a porogen is a decomposable material that is radiation, thermally, chemically or moisture decomposable, degradable, depolymerizable or otherwise capable of breaking down and includes solid, liquid or gaseous material.

In another aspect of the subject matter taught herein, the porogen may serve a dual purpose or multi-stage purpose. The porogen may be specifically chosen for a particular coating composition based on polarity and/or functional groups. Once the porogen is incorporated into the composition, either pre-bake (no significant pores/voids) or post-bake (pores/voids present in material), it will act effectively as a “magnet” to attract the stripping and/or etching solution to the porogen by utilizing a difference in polarity between the porogen or by utilizing the functional groups on the porogen. This attraction effect by the porogen can be activated in several ways. For example, there may be an exothermic reaction that takes place when the porogen is incorporated into the absorbing composition and/or coating material at room temperature, there may be an external energy and/or heat that needs to be added to “activate” the porogen or there may be a pressure differential applied to the absorbing composition and/or coating material that will “activate” the porogen. The porogen may be added to the composition as a material modification agent without ever intending the porogen to create pores and/or voids. If pores or voids are formed in the material, coating and/or film the pores/voids will create additional surface area in the coating or film which ultimately increases the etch selectivity and/or stripping selectivity of the material, coating and/or film, as described in the earlier embodiments.

In some contemplated embodiments, a decomposed porogen is removable from or can volatilize or diffuse through a partially or fully cross-linked matrix to create pores in a subsequently fully-cured matrix and thus, lower the matrix's dielectric constant and enhance the sacrificial properties. In another embodiment, the porogen might be a material, which does not decompose but can be dissolved out of the matrix leaving behind the “pore”. In a third embodiment the porogen might be a material that does not decompose but is volatile enough to dissipate at specific elevated temperatures such as in the 250-350° C. range. Supercritical materials, such as CO₂, may be used to remove the porogen and decomposed porogen fragments. Preferably, for a thermally decomposable porogen, the porogen comprises a material having a decomposition temperature greater than the minimum crosslinking temperature of the material. Preferably, the present novel porogens have a degradation or decomposition temperature of up to about 300° C., and in some cases greater than about 300° C. Preferably, the degraded or decomposed porogens volatilize at a temperature greater than the minimum cross-linking temperature of the material with which the porogen is combined. Preferably, the degraded or decomposed porogens volatilize at a temperature between about 500 to about 450° C.

Other suitable porogens suitable for use in contemplated embodiments include polymers, preferably those which contain one or more reactive groups, such as hydroxyl or amino. Within these general parameters, a suitable polymer porogen for use in the compositions and methods disclosed herein is, e.g. a polyalkylene oxide, a monoether of a polyalkylene oxide, a diether of a polyalkylene oxide, bisether of a polyalkylene oxide, an aliphatic polyester, an acrylic polymer, an acetal polymer, a poly(caprolactone), a poly(valeractone), a poly(methlymethoacrylate), a poly(vinylbutyral) and/or combinations thereof. When the porogen is a polyalkylene oxide monoether, one particular embodiment is a C₁ to about C₆ allyl chain between oxygen atoms and a C₁ to about C₆ alkyl ether moiety, and wherein the alkyl chain is substituted or unsubstituted, e.g., polyethylene glycol monomethyl ether, polyethylene glycol dimethyl ether, or polypropylene glycol monomethyl ether.

Porogens comprising at least two fused aromatic rings wherein each of the fused aromatic rings has at least one alkyl substituent thereon and a bond exists between at least two of the alkyl substituents on adjacent aromatic rings may be used in the present invention. Contemplated porogens include unfunctionalized polyacenaphthylene homopolymer, functionalized polyacenaphthylene homopolymer, the polyacenaphthylene copolymers described below, poly(2-vinylnaphthalene), and vinyl anthracene, and blends with each other. Other useful porogens include adamantane, diamantane, fullerene, and polynorbornene. Each of these porogens may be blended with each other or other porogen materials such as polycaprolactone, polystyrene, and polyester. Useful blends include unfunctionalized polyacenaphthylene homopolymer and polycaprolactone. The more contemplated porogens are unfunctionalized polyacenaphthylene homopolymer, functionalized polyacenaphthylene homopolymer, polyacenaphthylene copolymer, and polynorbornene.

Useful polyacenaphthylene homopolymers may have weight average molecular weights ranging from in contemplated embodiments about 300 to about 20,000; more in contemplated embodiments about 300 to about 10,000; and most in contemplated embodiments about 1000 to about 7,000 and may be polymerized from acenaphthylene using different initiators such as 2,2′-azobisisobutyronitrile (AIBN); di-tert-butyl azodicarboxylate; di-isopropyl azodicarboxylate; di-ethyl azodicarboxylate; di-benzyl azodicarboxylate; di-phenyl azodicarboxylate; 1,1′-azobis(cyclohexanecarbonitrile); benzoyl peroxide (BPO); t-butyl peroxide; and boron trifluoride diethyl etherate. The polyacenaphthylene homopolymer may have functional end groups such as triple bonds or double bonds to the chain end or cationic polymerization quenched with a double or triple bond alcohol such as allyl alcohol; propargyl alcohol; butynol; butenol; or hydroxyethylmethacrylate.

Useful polyacenaphthylene copolymers may be linear polymers, star polymers, or hyperbranched. The comonomer may have a bulky side group that will result in copolymer conformation that is similar to that of polyacenaphthylene homopolymer or a nonbulky side group that will result in copolymer conformation that is dissimilar to that of polyacenaphthylene homopolymer. Comonomers having a bulky side group include vinyl pivalate; tert-butyl acrylate; styrene; α-methylstyrene; tert-butylstyrene; 2-vinylnaphthalene; 5-vinyl-2-norbornene; vinyl cyclohexane; vinyl cyclopentane; 9-vinylanthracene; 4-vinylbiphenyl; tetraphenylbutadiene; stilbene; tert-butylstilbene; and indene; and in contemplated embodiments, vinyl pivalate. Hydridopolycarbosilane may be used as an additional co-monomer or copolymer component with acenaphthylene and at least one of the preceding comonomers. An example of a useful hydridopolycarbosilane has 10% or 75% allyl groups. Comonomers having a nonbulky side group include vinyl acetate; methyl acrylate; methyl methacrylate; and vinyl ether and in contemplated embodiments, vinyl acetate.

In contemplated embodiments, the amount of comonomer ranges from about 5 to about 50 mole percent of the copolymer. These copolymers may be made by free radical polymerization using initiator. Useful initiators include in contemplated embodiments 2,2′-azobisisobutyronitrile (AIBN); di-tert-butyl azodicarboxylate; di-isopropyl azodicarboxylate; di-ethyl azodicarboxylate; di-benzyl azodicarboxylate; di-phenyl azodicarboxylate; 1,1′-azobis(cyclohexanecarbonitrile); benzoyl peroxide (BPO); and t-butyl peroxide and more in contemplated embodiments, AIBN. Copolymers may also be made by cationic polymerization using initiator such as boron trifluoride diethyl etherate. In contemplated embodiments, the copolymers have a molecular weight from about 500 to about 15,000.

Thermal properties of copolymers of acenaphthylene and comonomers are set forth in the following Table 2. In Table 2, BA stands for butyl acrylate; VP stands for vinyl pivalate; VA stands for vinyl acetate; AIBN stands for 2,2′-azobisisobutyronitrile; BF₃ stands for boron trifluoride diethyl etherate; DBADC stands for di-tert-butyl azodicarboxylate; W1 stands for weight loss percentage from room temperature to 250° C.; W2 stands for weight loss percentage at 250° C. for 10 minutes; W3 stands for weight loss percentage from 250° C. to 400° C.; W4 stands for weight loss percentage at 400° C. for one hour; and W5 stands for total weight loss.

TABLE 2

| | | | Co- | | | | | | | | | | | |
|---|---|---|---|---|---|---|---|---|---|---|---|---|---|---|
| Co- | | | mono. | Init. | | Temp. | Time | | | | | | | |
| mono. | Copoly. | Init. | % | % | Solvent | (° C.) | (hr) | W1 | W2 | W3 | W4 | W5 | Mn | Mw |
| BA | 1 | AIBN | 11 | 1 | Xylene | 70 | 24 | 14.63 | 1.02 | 33.14 | 30.44 | 79.23 | 4797 | 10552 |
| BA | 2 | AIBN | 20 | 1 | Xylene | 70 | 24 | 1.47 | 0.98 | 37.92 | 35.55 | 75.92 | 4343 | 8103 |
| BA | 3 | AIBN | 30 | 1 | Xylene | 70 | 24 | 13.41 | 1.6 | 36.48 | 27.55 | 79.04 | 4638 | 7826 |
| BA | 4 | AIBN | 50 | 1 | Xylene | 70 | 24 | 10.01 | 2.96 | 46.92 | 26.51 | 86.40 | 3504 | 5489 |
| BA | 5 | BF3 | 10 | 3 | Xylene | 5 | 2 | 11.93 | 0.58 | 40.06 | 29.33 | 81.90 | 1502 | 2421 |
| VP | 6 | AIBN | 10 | 1 | Xylene | 70 | 24 | 16.22 | 0.41 | 37.8 | 34.72 | 89.15 | 5442 | 10007 |
| VP | 7 | AIBN | 16 | 1 | THF | 60 | 12 | 5.32 | 0.66 | 46.55 | 29.59 | 82.12 | 1598 | 2422 |
| VP | 8 | AIBN | 25 | 1 | Xylene | 70 | 24 | 4.15 | 0.37 | 24.98 | 47.4 | 76.90 | 2657 | 8621 |
| VP | 9 | AIBN | 30 | 1 | Xylene | 70 | 24 | 14.7 | 0.69 | 33.27 | 39.54 | 88.20 | 5342 | 9303 |
| VP | 10 | AIBN | 40 | 1 | Xylene | 70 | 24 | 6.34 | 0.26 | 33.69 | 39.38 | 76.67 | 4612 | 7782 |
| VP | 11 | AIBN | 50 | 1 | Xylene | 70 | 24 | 14.12 | 0.32 | 29.01 | 37.86 | 81.31 | 4037 | 6405 |
| VP | 12 | BF3 | 10 | 1 | Xylene | 5 | 2 | 0.84 | 0 | 55.51 | 39.38 | 95.73 | 2078 | 3229 |
| VP | 13 | BF3 | 10 | 3 | Xylene | 5 | 2 | 2.26 | 0.06 | 47.44 | 28.93 | 78.69 | 1786 | 2821 |
| VP | 14 | BF3 | 25 | 1 | Xylene | 5 | 2 | 0.17 | 0 | 36.99 | 41.17 | 78.33 | 2381 | 3549 |
| VP | 15 | BF3 | 25 | 3 | Xylene | 5 | 2 | 1.33 | 0.03 | 35.28 | 41.08 | 77.72 | 2108 | 3267 |
| VP | 16 | BF3 | 40 | 1 | Xylene | 5 | 2 | 0.23 | 0.04 | 36.46 | 42.17 | 78.90 | 2659 | 3692 |
| VP | 17 | BF3 | 40 | 3 | Xylene | 5 | 2 | 0.28 | 0.01 | 40.23 | 38.98 | 79.50 | 2270 | 3376 |
| VA | 18 | AIBN | 20 | 2 | Xylene | 70 | 24 | 16.93 | 1.346 | 38.42 | 21.43 | 78.13 | 3404 | 7193 |
| VA | 19 | AIBN | 40 | 2 | Xylene | 70 | 24 | 15.45 | 1.631 | 31.28 | 31.64 | 80.00 | 3109 | 6141 |

Known porogens such as linear polymers, star polymers, crosslinked polymeric nanospheres, block copolymers, and hyperbranched polymers may be used with the novel thermosetting component described above. Suitable linear polymers are polyethers such as poly(ethylene oxide) and polypropylene oxide); polyacrylates such as poly(methylmethacrylate); aliphatic polycarbonates such as poly(propylene carbonate) and poly(ethylene carbonate); polyesters; polysulfones; polystyrene (including monomer units selected from halogenated styrene and hydroxy-substituted styrene); poly(α-methylstyrene); polylactides; and other vinyl based polymers. Useful polyester porogens include polycaprolactone; polyethylene terephthalate; poly(oxyadipoyloxy-1,4-phenylene); poly(oxyterephthaloyloxy-1,4-phenylene); poly(oxyadipoyloxy-1,6-hexamethylene); polycarbonate such as poly(hexamethylene carbonate) diol having a molecular weight from about 500 to about 2500; and polyether such as poly(bisphenol A-co-epichlorohydrin) having a molecular weight from about 300 to about 6,500. Suitable crosslinked, insoluble nanospheres (prepared as nanoemulsions) are suitably comprised of polystyrene or poly(methylmethacrylate). Suitable block copolymers are poly(styrene-corct-methylstyrene), poly(styrene-ethylene oxide), poly(etherlactones), poly(estercarbonates), and poly(lactonelactide). Suitable hyperbranched polymers are hyperbranched polyester, e.g., hyperbranched poly(caprolactone), and polyethers such as polyethylene oxide and polypropylene oxide. Another useful porogen is ethylene glycol-poly(caprolactone). Useful polymer blocks include polyvinylpyridines, hydrogenated polyvinyl aromatics, polyacrylonitriles, polysiloxanes, polycaprolactams, polyurethanes, polydienes such as polybutadienes and polyisoprenes, polyvinyl chlorides, polyacetals, and amine-capped alkylene oxides. Other useful thermoplastic materials include polyisoprenes, polytetrahydrofurans, and polyethyloxazolines.

When discussing pore generation, the term “degrade” refers to the breaking of covalent bonds. Such breaking of bonds may occur in numerous ways including heterolytic and homolytic breakage. The breaking of bonds need not be complete, i.e., not all breakable bonds must be cleaved. Furthermore, the breaking of bonds may occur in some bonds faster than in others. Ester bonds, for example, are generally less stable than amide bonds, and therefore, are cleaved at a faster rate. Breakage of bonds may also result in the release of fragments differing from one another, depending on the chemical composition of the degraded portion.

In a preferred embodiment of the pore generation process, for thermally degradable porogens, thermal energy is applied to the porogen containing material to substantially degrade or decompose the porogen into its starting components or monomers. As used herein, “substantially degrade” preferably means at least about 40 weight percent of the porogen degrades or decomposes. In more preferred embodiments, at least about 50 weight percent of the porogen degrades or decomposes, and in most preferred embodiments, at least about 80 weight percent of the porogen degrades or decomposes. In another embodiment, the porogen is dissolved out in either a separate process stage or in combination with other stages of process, such as during the photolithography development or during the actual wet stripping of the porogen containing material.

For a contemplated embodiment, thermal energy is also applied to volatilize the substantially degraded or decomposed porogen out of the inorganic compound matrix. Preferably, the same thermal energy is used for both the degradation and volatilization steps. As the amount of volatilized degraded porogen increases, the resulting porosity of the material, coating and/or film increases.

Any suitable procedure or condition may be used to remove or at least partially remove the at least one porogen, including heat, dissolution in solvents, preferential etching, exposure to radiation, electromagnetic radiation, such as ultraviolet, x-ray, laser or infrared radiation; mechanical energy, such as sonication or physical pressure; or particle radiation, such as gamma ray, alpha particles, neutron beam or electron beam as taught by commonly assigned patent publication PCT/US96/08678 and U.S. Pat. Nos. 6,042,994; 6,080,526; 6,177,143; and 6,235,353, which are incorporated herein by reference in their entireties.

In contemplated embodiments, for a thermally decomposable porogen, the porogen comprises a material having a decomposition temperature less than the glass transition temperature (Tg) of a material combined with it and greater than the curing temperature of the material combined with it.

In one embodiment, the porogen bonds to the thermosetting component. In contemplated embodiments, the porogens have a degradation or decomposition temperature of about 350° C. or greater. In contemplated embodiments, the degraded or decomposed porogens volatilize at a temperature greater than the cure temperature of the material with which the porogen is combined and less than the Tg of the material. In contemplated embodiments, the degraded or decomposed porogens volatilize at a temperature of about 96° C. or greater.

The phrase “porogen bonds to the thermosetting component” covers addition reactions, nucleophilic and electrophilic substitutions or eliminations, radical reactions, etc. Further alternative reactions may also include the formation of non-covalent bonds, such as Van der Waals, electrostatic bonds, ionic bonds, and hydrogen bonds.

In some contemplated embodiments, porogens comprise unsubstituted polynorbornene, substituted polynorbornene, polycaprolactone, unsubstituted polystyrene, substituted polystyrene, polyacenaphthylene homopolymer, and polyacenaphthylene copolymer. The more contemplated porogen is substituted polynorbornene. In contemplated embodiments, the porogen has functional groups selected from the group consisting of epoxy, hydroxy, carboxylic acid groups, amino, and ethynyl. In contemplated embodiments, the porogen has a functional group on at least one of its ends.

In contemplated embodiments, the porogen is bonded to the thermosetting component through an ethynyl containing group. In one embodiment, the ethynyl containing group is first reacted with the porogen. In a contemplated embodiment, the ethynyl containing group is first reacted with the thermosetting component. Useful ethynyl containing groups include fluorine; amine; or hydroxy; and in contemplated embodiments, are acetylene; 4-ethynylaniline; 3-hydroxyphenylacetylene; 4-fluorophenylacetylene; and 1-ethylcyclohexylamine. In contemplated embodiments, a covalent bond forms between the porogen and the thermosetting component through the ethynyl containing group.

Useful polyacenaphthylene homopolymers may have weight average molecular weights ranging from in contemplated embodiments about 300 to about 20,000; more in contemplated embodiments about 300 to about 10,000; and most in contemplated embodiments about 300 to about 7,000.

The amount of thermosetting component used is about 50 to about 90 weight percent while the amount of porogen used is about 10 to about 50 weight percent. In contemplated embodiments, an adhesion promoter as described above is added to the porogen bonded to the thermosetting component. Based on a composition comprising the adhesion promoter and the porogen bonded to the thermosetting component, about 0.1 to about 15 weight percent of adhesion promoter is used and about 5 to about 50 weight percent porogen bonded to the thermosetting component is used.

In another embodiment, the porogen and thermosetting component form a physical mixture. In contemplated embodiments, the porogens have a degradation or decomposition temperature of about 350° C. or greater. In contemplated embodiments, the degraded or decomposed porogens volatilize at a temperature greater than the cure temperature of the material with which the porogen is combined and less than the Tg of said material. In contemplated embodiments, the degraded or decomposed porogens volatilize at a temperature of about 280° C. or greater.

Pore Generation:

As mentioned, the term “degrade” as used herein refers to the breaking of covalent bonds. Such breaking of bonds may occur in numerous ways including heterolytic and homolytic breakage. The breaking of bonds need not be complete, i.e., not all breakable bonds must be cleaved. Furthermore, the breaking of bonds may occur in some bonds faster than in others. Ester bonds, for example, are generally less stable than amide bonds, and therefore, are cleaved at a faster rate. Breakage of bonds may also result in the release of fragments differing from one another, depending on the chemical composition of the degraded portion.

In the pore generation process, for thermally degradable porogens, thermal energy is applied to the porogen containing material to substantially degrade or decompose the porogen into its starting components or monomers. As used herein, “substantially degrade” in contemplated embodiments means at least 80 weight percent of the porogen degrades or decomposes. For the contemplated thermosetting component of Formulae I and II above, the Tg is from about 400° C. to about 450° C. so the present porogens which have a degradation or decomposition temperature of about 350° C. or greater are particularly useful with this thermosetting component. For the contemplated polyacenaphthylene based homopolymer or copolymer porogen, we have found by using analytical techniques such as Thermal Desorption Mass Spectroscopy that the porogen degrades, decomposes, or depolymerizes into its starting components of acenaphthylene monomer and comonomer.

Thermal energy is also applied to volatilize the substantially degraded or decomposed porogen out of the thermosetting component matrix. In contemplated embodiments, the same thermal energy is used for both the degradation and volatilization steps. As the amount of volatilized degraded porogen increases, the resulting porosity of the thermosetting component increases. For the contemplated thermosetting components of Formulae I and II above, the Tg is from about 400° C. to about 450° C. so the present substantially degraded porogens which have a volatilization temperature of about 280° C. or greater are particularly useful with the thermosetting component.

In contemplated embodiments, the cure temperature used for cross-linking the thermosetting component will also substantially degrade the porogen and volatilize it out of the thermosetting matrix. Typical cure temperature and conditions will be described in the “Utility” section below.

The resulting pores may be uniformly or randomly dispersed throughout the matrix. In contemplated embodiments, the pores are uniformly dispersed throughout the matrix.

Alternatively, other procedures or conditions which at least partially remove the porogen without adversely affecting the thermosetting component may be used. In contemplated embodiments, the porogen is substantially removed. Typical removal methods include, but are not limited to, exposure to radiation, such as but not limited to, electromagnetic radiation such as ultraviolet, x-ray, laser, or infrared radiation; mechanical energy such as sonication or physical pressure; or particle radiation such as gamma ray, alpha particles, neutron beam, or electron beam.

Utility:

Each of the compositions set forth above may be processed and used as disclosed below. Each of the present compositions may also comprise additional components such as adhesion promoters, antifoam agents, detergents, flame retardants, pigments, plasticizers, stabilizers, striation modifiers, and surfactants.

The present composition may be combined with other specific additives to obtain specific results. Representative of such additives are metal-containing compounds such as magnetic particles, for example, barium ferrite, iron oxide, optionally in a mixture with cobalt, or other metal containing particles for use in magnetic media, optical media, or other recording media; conductive particles such as metal or carbon for use as conductive sealants, conductive adhesives, conductive coatings, electromagnetic interference (EMI)/radio frequency interference (RFI) shielding coating, static dissipation, and electrical contacts. When using these additives, the present compositions may act as a binder. The present compositions may also be employed as protection against manufacturing, storage, or use environment such as coatings to impart surface passivation to metals, semiconductors, capacitors, inductors, conductors, solar cells, glass and glass fibers, quartz, and quartz fibers.

The present composition is also useful in anti-fouling coatings on such objects as boat parts; electrical switch enclosures; bathtubs and shower coatings; in mildew resistant coatings; or to impart flame resistance, weather resistance, or moisture resistance to an article. Because of the range of temperature resistance of the present compositions, the present compositions may be coated on cryogenic containers, autoclaves, and ovens, as well as heat exchanges and other heated or cooled surfaces and on articles exposed to microwave radiation.

The present composition is particularly useful as a dielectric material. The dielectric material has a dielectric constant of in contemplated embodiments less than or equal to about 3.0 and more in contemplated embodiments from about 2.3 to 3.0. The dielectric material has a glass transition temperature of in contemplated embodiments at least about 350° C.

A contemplated method of forming a coating solution comprises: a) providing at least one of the compositions described herein; b) providing at least one solvent and c) combining the at least one composition with the at least one solvent to form the solution. In additional methods, at least one other component, such as an adhesion promoter, a porogen, or another component such as those previously described, may be provided and combined with the at least one composition and the at least one solvent to form the solution.

Layers of the instant compositions may be formed by solution techniques such as spraying, rolling, dipping, spin coating, flow coating, or casting, with spin coating being contemplated for microelectronics. Suitable solvents for use in such solutions of the present compositions of the present invention include any suitable pure or mixture of organic, organometallic, or inorganic molecules that are volatized at a desired temperature. Typical solvents are also those solvents that are able to solvate the monomers and polymers contemplated herein to be used as coating compositions and materials. Contemplated solvents include any suitable pure or mixture of organic, organometallic or inorganic molecules that are volatilized at a desired temperature. The solvent may also comprise any suitable pure or mixture of polar and non-polar compounds.

Suitable solvents include aprotic solvents, for example, cyclic ketones such as cyclopentanone, cyclohexanone, cycloheptanone, and cyclooctanone; cyclic amides such as N-alkylpyrrolidinone wherein the alkyl has from about 1 to 4 carbon atoms; and N-cyclohexylpyrrolidinone and mixtures thereof. A wide variety of other organic solvents may be used herein insofar as they effectively control the viscosity of the resulting solution as a coating solution. Various facilitating measures such as stirring and/or heating may be used to aid in the dissolution. Other suitable solvents include methyethylketone, methylisobutylketone, dibutyl ether, cyclic dimethylpolysiloxanes, butyrolactone, γ-butyrolactone, 2-heptanone, ethyl 3-ethoxypropionate, polyethylene glycol [di]methyl ether, propylene glycol methyl ether acetate (PGMEA), anisole, and hydrocarbon solvents such as mesitylene, xylenes, benzene, and toluene. A contemplated solvent is cyclohexanone. Typically, layer thicknesses are between 0.1 to about 15 microns. As a dielectric interlayer for microelectronics, the layer thickness is generally less than 2 microns. The amount of solvent added to the composition is at least about 70 weight percent.

In contemplated embodiments, the present composition is dissolved in solvent and treated at a temperature from about 30° C. to about 350° C. for about 0.5 to about 60 hours.

The compositions disclosed herein may be applied to various substrates and/or surfaces to form sacrificial via fill layers, layered materials, layers used in semiconductor processing, or layers used in electronic components, depending on the specific fabrication process, typically by conventional spin-on deposition techniques, vapor deposition or chemical vapor deposition.

The present composition may be used as an interlayer dielectric in an interconnect typically have on its surface a plurality of layers of the instant composition and multiple layers of metal conductors. It may also include regions of the present composition between discrete metal conductors or regions of conductor in the same layer or level of an integrated circuit.

Contemplated coating materials, coating solutions and films can be utilized are useful in the fabrication of a variety of electronic devices, micro-electronic devices, particularly semiconductor integrated circuits and various layered materials for electronic and semiconductor components, including hardmask layers, dielectric layers, etch stop layers and buried etch stop layers. These coating materials, coating solutions and films are quite compatible with other materials that might be used for layered materials and devices, such as adamantane-based compounds, diamantane-based compounds, silicon-core compounds, organic dielectrics, and nanoporous dielectrics. Compounds that are considerably compatible with the coating materials, coating solutions and films contemplated herein are disclosed in PCT Application PCT/US01/32569 filed Oct. 17, 2001; PCT Application PCT/US01/50812 filed Dec. 31, 2001; U.S. application Ser. No. 09/538,276; U.S. application Ser. No. 09/544,504; U.S. application Ser. No. 09/587,851; U.S. Pat. No. 6,214,746; U.S. Pat. No. 6,171,687; U.S. Pat. No. 6,172,128; U.S. Pat. No. 6,156,812, U.S. application Ser. No. 60/350187 filed Jan. 15, 2002; and U.S. 60/347195 filed Jan. 8, 2002, which are all incorporated herein by reference in their entirety.

The compounds, coatings, films, materials and the like described herein may be used to become a part of, form part of or form an electronic component and/or semiconductor component. As used herein, the term “electronic component” also means any device or part that can be used in a circuit to obtain some desired electrical action. Electronic components contemplated herein may be classified in many different ways, including classification into active components and passive components. Active components are electronic components capable of some dynamic function, such as amplification, oscillation, or signal control, which usually requires a power source for its operation. Examples are bipolar transistors, field-effect transistors, and integrated circuits. Passive components are electronic components that are static in operation, i.e., are ordinarily incapable of amplification or oscillation, and usually require no power for their characteristic operation. Examples are conventional resistors, capacitors, inductors, diodes, rectifiers and fuses.

Electronic components contemplated herein may also be classified as conductors, semiconductors, or insulators. Here, conductors are components that allow charge carriers (such as electrons) to move with ease among atoms as in an electric current. Examples of conductor components are circuit traces and vias comprising metals. Insulators are components where the function is substantially related to the ability of a material to be extremely resistant to conduction of current, such as a material employed to electrically separate other components, while semiconductors are components having a function that is substantially related to the ability of a material to conduct current with a natural resistivity between conductors and insulators. Examples of semiconductor components are transistors, diodes, some lasers, rectifiers, thyristors and photosensors.

Electronic components contemplated herein may also be classified as power sources or power consumers. Power source components are typically used to power other components, and include batteries, capacitors, coils, and fuel cells. Power consuming components include resistors, transistors, integrated circuits (ICs), sensors, and the like.

Still further, electronic components contemplated herein may also be classified as discreet or integrated. Discreet components are devices that offer one particular electrical property concentrated at one place in a circuit. Examples are resistors, capacitors, diodes, and transistors. Integrated components are combinations of components that that can provide multiple electrical properties at one place in a circuit. Examples are integrated circuits in which multiple components and connecting traces are combined to perform multiple or complex functions such as logic.

In application of the instant polymers to ICs, a solution of the present composition is applied to a semiconductor wafer using conventional wet coating processes as, for example, spin coating; other well known coating techniques such as spray coating, flow coating, or dip coating may be employed in specific cases. As an illustration, a cyclohexanone solution of the present composition is spin-coated onto a substrate having electrically conductive components fabricated therein and the coated substrate is then subjected to thermal processing. The present composition may be used in substractive metal (such as aluminum and aluminum/tungsten) processing and dual damascene (such as copper) processing. An exemplary formulation of the instant composition is prepared by dissolving the present composition in cyclohexanone solvent under ambient conditions with strict adherence to a clean-handling protocol to prevent trace metal contamination in any conventional apparatus having a non-metallic lining. The resulting solution comprises based on the total solution weight, from in contemplated embodiments about 1 to about 50 weight percent of the present composition and about 50 to about 99 weight percent solvent and more in contemplated embodiments from about 3 to about 30 weight percent of the present composition and about 70 to about 97 weight percent solvent.

An illustration of the use of the compositions described herein follows. A solvent solution of the present composition is provided in an amount of from about 5 to about 10 weight percent (%) based on the composition. Application of the instant compositions onto planar or topographical surfaces or substrates may be carried out by using any conventional apparatus, in contemplated embodiments a spin coater, because the compositions used herein have a controlled viscosity suitable for such a coater. Complete evaporation of the solvent by any suitable means, such as simple air drying during spin coating, by exposure to an ambient environment, or by heating on a hot plate up to 350° C., may be employed. The substrate may have on it at least one layer of the present composition.

The compositions disclosed herein may also be used as a dielectric substrate material in microchips, multichip modules, laminated circuit boards, or printed wiring boards. The circuit board made up of the present composition will have mounted on its surface patterns for various electrical conductor circuits. The circuit board may include various reinforcements, such as woven non-conducting fibers or glass cloth. Such circuit boards may be single sided, as well as double sided.

After application of the present composition to an electronic topographical substrate, the coated structure is subjected to a bake and cure thermal process at increasing temperatures ranging from about 50° C. up to about 450° C. to polymerize the coating. The contemplated curing temperature is at least about 300° C. Generally, it is contemplated that curing is carried out at temperatures of from about 350° C. to about 425° C. Curing may be carried out in a conventional curing chamber such as an electric furnace, hot plate, and the like and is generally performed in an inert (non-oxidizing) atmosphere (nitrogen) in the curing chamber. In addition to furnace or hot plate curing, the present compositions may also be cured by exposure to ultraviolet radiation, microwave radiation, or electron beam radiation as taught by commonly assigned patent publication PCT/US96/08678 and U.S. Pat. Nos. 6,042,994; 6,080,526; 6,177,143; and 6,235,353, which are incorporated herein by reference in their entireties. Any non-oxidizing or reducing atmospheres (eg. argon, helium, hydrogen, and nitrogen processing gases) may be used in the practice of the present invention.

As indicated earlier, the present coating may act as an interlayer and be on top of or covered by other coatings, such as other dielectric (SiO₂) coatings, SiO₂ modified ceramic oxide layers, silicon containing coatings, silicon carbon containing coatings, silicon nitrogen containing coatings, silicon-nitrogen-carbon containing coatings, diamond like carbon coatings, titanium nitride coatings, tantalum nitride coatings, tungsten nitride coatings, aluminum coatings, copper coatings, tantalum coatings, organosiloxanes coatings, organo silicon glass coatings, and fluorinated silicon glass coatings. Such multilayer coatings are taught in U.S. Pat. No. 4,973,526, which is incorporated herein by reference. And, as amply demonstrated, the present compositions prepared in the instant process may be readily formed as interlined dielectric layers between adjacent conductor paths on fabricated electronic or semiconductor substrates.

The present compositions are advantageous in that in contemplated embodiments, they are capable of generating films having thicknesses as thin as 50 Angstroms or as thick as ≧1.0 micron (10,000 Angstroms) and even ≧1.5 microns (15,000 Angstroms). Thus, contemplated layers of the present compositions have a thickness up to or greater than about 1.5 microns.

The present films may be used in dual damascene (such as copper) processing and substractive metal (such as aluminum or aluminum/tungsten) processing for integrated circuit manufacturing. The present compositions may be used as an etch stop, hardmask, air bridge, or passive coating for enveloping a completed wafer. The present composition may be used in a desirable all spin-on stacked film as taught by Michael E. Thomas, “Spin-On Stacked Films for Low k_{eff} Dielectrics”, Solid State Technology (July 2001), incorporated herein in its entirety by reference. The present layers may be used in stacks with other layers comprising organosiloxanes such as taught by commonly assigned U.S. Pat. No. 6,143,855 and pending U.S. Ser. No. 10/078,919 filed Feb. 19, 2002; Honeywell International Inc.'s commercially available HOSP® product; nanoporous silica such as taught by commonly assigned U.S. Pat. No. 6,372,666; Honeywell International Inc.'s commercially available NANOGLASS® E product; organosilsesquioxanes taught by commonly assigned U.S. Pat. No. 6,472,076; and fluorosilsesquioxanes taught by commonly assigned U.S. Pat. No. 6,440,550, incorporated herein in their entirety.

### EXAMPLES

Analytical Test Methods:

Gel Permeation Chromatography (GPC 1):

This analysis was done with Waters liquid chromatography system composed from Water 717 plus Autosampler, Waters in-line degasser, Waters 515 HPLC pump, Waters 410 Differential Refractometer (RI detector), and two columns: HP P1 gel 5μ MIXED D. The analysis conditions were:

| Mobile Phase | Tetrahydrofuran (THF) |
|---|---|
| Column flow (milliliters/min) | 1.0 |
| Column temperature (° C.) | 40.0 |
| Detection | Refractive Index, Polarity positive |
| Analysis run time | 25 min |
| Injection quantity (μL) | 50 |

10 milligrams of solid product were dissolved in one milliliter of tetrahydrofuran. To calculate the contents, in other words area-%, the peak area belonging to the monomer or the peak area belonging to the oligomer or polymer is related to the total of all the peak areas in the chromatogram.

Gel Permeation Chromatography (GPC 2):

This method may be used to provide additional detail about the dimer and trimer peaks. The following conditions are used:

| Analysis Apparatus | Shimadzu LC10 |
|---|---|
| Separation Column | Plgel 5 μ pre-column |
| | Plgel 5 μ 1000 Å, 300 × 7.5 nm |
| | Plgel 5 μ 500 Å, 300 × 7.5 nm |
| | Plgel 5 μ 100 Å, 300 × 7.5 nm |
| Mobile Phase | Eluent A: toluene |
| Column Flow (milliliters/minute) | 1.0 |
| Column Temperature (° C.) | 40 |
| Detection | Refractive Index, Polarity Positive |
| Analysis Run Time (minutes) | 32 |
| Trial Solution | 10 milligrams/milliliter toluene |
| Injection Quantity (μ) | 50 |

To calculate the contents, in other words area-%, the peak area belonging to the monomer or the peak area belonging to the oligomer or polymer is related to the total of all the peak areas in the chromatogram.

Gel Permeation Chromatography (GPC 3):

Separation was performed with a Waters 2690 separation module with Waters 996 diode array and Waters 410 differential refractometer detectors. The separation was performed on two PLgel 3 μm Mixed-E 300×7.5 mm columns with chloroform flowing at 1 ml/min. Injection volumes of 25 μl of solutions of about 1 mg/ml concentration were run in duplicate. Good reproducibility was observed.

The column was calibrated with relatively monodisperse polystyrene standards between 20,000 and 500 molecular weight. With the lower molecular weight standards nine distinct components could be resolved corresponding to butyl terminated styrene monomer through oligomers with nine styrenes. The logs of the peak molecular weight of the standards were fit with a third order polynomial of the elution time. The instrumental broadening was evaluated from the ratio of the full width at half maximum to the mean elution time of toluene.

The absorbance for Preparations 1 and 2 below was a maximum at about 284 nm. The chromatograms had similar shapes at absorbance at wavelengths below about 300 nm. The results presented here correspond to 254 nm absorbance. The peaks were identified by the molecular weight of the polystyrene that would be eluting at the same time. These values should not be considered as measurements of molecular weight of the Preparation 1 and 2 oligomers. The sequential elution of higher oligomers, trimers, dimers, oligomers, and incomplete oligomers at increasing times can be quantitated.

Each component was broader than that which would be observed for a monodisperse species. This width was analyzed from the full width in minutes at half maximum of the peak. To roughly account for the instrumental broadening, we calculated
width_{corrected}=[width_{observed}²−widthᵢₙₛₜᵣᵤₘₑₙₜ²]^{1/2}
where widthᵢₙₛₜᵣᵤₘₑₙₜ is the observed width of toluene corrected by the ratio of the elution times of the peal( to that for toluene. The peak width was converted to a molecular weight width through the calibration curve and ratioed to the peak molecular width. Since the molecular weight of styrene oligomers was proportional to the square of their size, the relative molecular weight width can be converted to a relative oligomer size width by dividing by 2. This procedure accounted for the difference in molecular configuration of the two species.
Proton NMR:

A 2-5 mg sample of the material to be analyzed was put into an NMR tube. About 0.7 ml deuterated chloroform was added. The mixture was shaken by hand to dissolve the material. The sample was then analyzed using a Varian 400 MHz NMR.

High Performance Liquid Chromatography (HPLC):

A HPLC with a Phenomenex luna Phenyl-Hexyl 250×4.6 mm 5 micron column was used. The column temperature was set at 40° C. Water and acetonitrile were used to improve peak separation as the eluent.

| TIME | WATER | ACETONITRILE |
|---|---|---|
| Initial | 20% | 80% |
| 10 minutes | 0% | 100% |
| 30 minutes | 0% | 100% |

The following experimental conditions were used:

| | |
|---|---|
| INJECTION VOLUME | 10 microliters |
| DETECTION | UV at 200 nm |
| STOP TIME | 30 minutes |
| POST TIME | 5 minutes |

The sample was prepared as follows. The sample was dissolved in tetrahydrofuran (THF) with 1 ml THF used per 1 mg of solid sample.

Liquid Chromatography-Mass Spectroscopy (LC-MS):

This analysis was performed on a Finnigan/MAT TSQ7000 triple stage quadrupole mass spectrometer system, with an Atmospheric Pressure Ionization (API) interface unit, using a Hewlett-Packard Series 1050 HPLC system as the chromatographic inlet. Both mass spectral ion current and variable single wavelength UV data were acquired for time-intensity chromatograms.

Chromatography was conducted on a Phenomenex Luna 5-micron pheny-hexyl column (250×4.6 mm). Sample auto-injections were generally between 5 and 20 microliters of concentrated solutions, both in tetrahydrofuran and without tetrahydrofuran. The contemplated preparation of concentrated sample solutions for analysis was dissolution in tetrahydrofuran, of about 5 milligrams solid product per milliliter, for 10 microliter injections. The mobile phase flow through the column was 1.0 milliliter/minute of acetonitrile/water, initially 70/30 for 1 minute then gradient programmed to 100% acetonitrile at 10 minutes and held until 40 minutes.

Atmospheric Pressure Chemical Ionization (APCI) mass spectra were recorded in both positive and negative ionization, in separate experiments. Positive APCI was more informative of molecular structure for these final products, providing protonated pseudomolecular ions including adducts with acetonitrile matrix. The APCI corona discharge was 5 microamps, about 5 kV for positive ionization, and about 4 kV for negative ionization.

The heated capillary line was maintained at 200° C. and the vaporizer cell at 400° C. The ion detection system after quadrupole mass analysis was set at 15 kV conversion dynode and 1500V electron multiplier voltage. Mass spectra were typically recorded at 1.0 second/scan from about m/z 50 to 2000 a.m.u. for negative ionization, and from about m/z 150 a.m.u. up for positive ionization. In separate positive ion experiments, the mass range was scanned up both to 2000 a.m.u. in low mass tune/calibration mode and to 4000 a.m.u. in high mass tune/calibration mode.

Differential Scanning Calorimetry (DSC):

DSC measurements were performed using a TA Instruments 2920 Differential Scanning Calorimeter in conjunction with a controller and associated software. A standard DSC cell with temperature ranges from 250 degree C. to 725 degree C. (inert atmosphere: 50 ml/min of Nitrogen) was used for the analysis. Liquid nitrogen was used as a cooling gas source. A small amount of sample (10-12 mg) was carefully weighed into an Auto DSC aluminum sample pan (Part #990999-901) using a Mettler Toledo Analytical balance with an accuracy of ±0.0001 grams. Sample was encapsulated by covering the pan with the lid that was previously punctured in the center to allow for outgasing. Sample was heated under nitrogen from 0° C. to 450° C. at a rate of 100° C./minute (cycle 1), then cooled to 0° C. at a rate of 100° C./minute. A second cycle was run immediately from 0° C. to 450° C. at a rate of 100° C./minute (repeat of cycle 1). The cross-linking temperature was determined from the first cycle.

Dielectric Constant:

The dielectric constant was determined by coating a thin film of aluminum on the cured layer and then doing a capacitance-voltage measurement at 1 MHz and calculating the k value based on the layer thickness.

Glass Transition Temperature (Tg):

The glass transition temperature of a thin film was determined by measuring the thin film stress as a function of temperature. The thin film stress measurement was performed on a KLA 3220 Flexus. Before the film measurement, the uncoated wafer was annealed at 500° C. for 60 minutes to avoid any errors due to stress relaxation in the wafer itself. The wafer was then deposited with the material to be tested and processed through all required process steps. The wafer was then placed in the stress gauge, which measures the wafer bow as function of temperature. The instrument can calculate the stress versus temperature graph, provided that the wafer thickness and the film thickness are known. The result was displayed in graphic form. To determine the Tg value, a horizontal tangent line was drawn (a slope value of zero on the stress vs. temperature graph). Tg value was where the graph and the horizontal tangent line intersect.

It should be reported if the Tg was determined after the first temperature cycle or a subsequent cycle where the maximum temperature was used, the measurement process itself mayinfluence Tg.

Shrinkage:

Film shrinkage was measured by determining the film thickness before and after the process. Shrinkage was expressed in percent of the original film thickness. Shrinkage was positive if the film thickness decreased. The actual thickness measurements were performed optically using a J. A. Woollam M-88 spectroscopic ellipsometer. A Cauchy model was used to calculate the best fit for Psi and Delta (details on Ellipsometry can be found in e.g. “Spectroscopic Ellipsometry and Reflectometry” by H. G. Thompkins and William A. McGahan, John Wiley and Sons, Inc., 1999).

Refractive Index:

The refractive index measurements were performed together with the thickness measurements using a J. A. Woollam M-88 spectroscopic ellipsometer. A Cauchy model was used to calculate the best fit for Psi and Delta. Unless noted otherwise, the refractive index was reported at a wavelenth of 633 nm (details on Ellipsometry can be found in e.g. “Spectroscopic Ellipsometry and Reflectometry” by H. G. Thompkins and William A. McGahan, John Wiley and Sons, Inc., 1999).

FTIR Analysis:

FTIR spectra were taken using a Nicolet Magna 550 FTIR spectrometer in transmission mode. Substrate background spectra were taken on uncoated substrates. Film spectra were taken using the substrate as background. Film spectra were then analyzed for change in peak location and intensity.

Compatibility with Solvents:

Compatibility with solvents was determined by measuring film thickness, refractive index, FTIR spectra, and dielectric constant before and after solvent treatment. For a compatible solvent, no significant change should be observed.

Solubility Improvement:

In a first vessel, product was added to cyclohexanone until visual inspection revealed that additional product would not be soluble in the cyclohexanone. The amount of solids added was recorded.

### Comparative Example 1

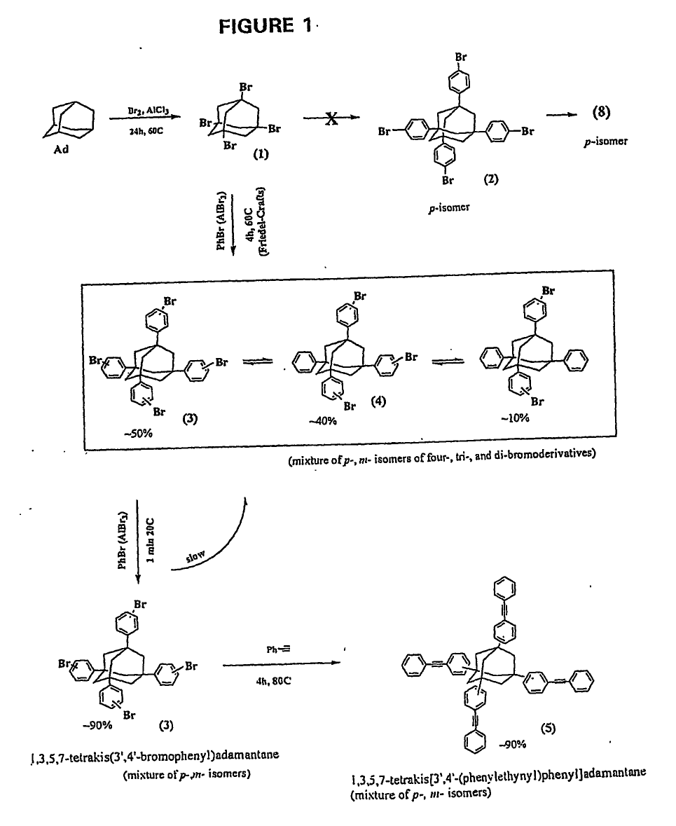

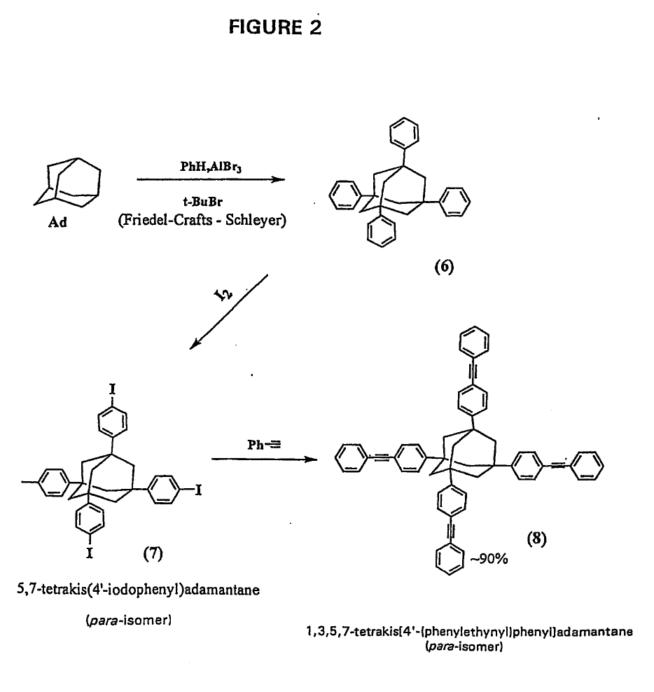

FIGS. 1 and 2 show the preparation of the isomers discussed below, and the Roman numerals in the text of this Example correspond with the Roman numerals in FIGS. 1 and 2. As mentioned briefly in the Background section, Reichert's goal was to prepare 1,3,5,7-tetrakis[(4-phenylethynyl)phenyl)]adamantane of definite structure, namely, single p-isomer of this compound—1,3,5,7-tetrakis[4′-(phenylethynyl)phenyl]adamantane (8). This, and only this compound, having definite structure (which can be characterized by the analytical methods) was the target of Reichert's work.

Reichert's plan was to realize the following sequence:
1,3,5,7-tetrabromoadamantane (1)→1,3,5,7-tetrakis(4′-bromophenyl)adamantane (2) p-isomer)→1,3,5,7-tetrakis[4′-(phenylethynyl)phenyl)]adamantane (8) (p-isomer)

Reichert failed on step (1)→(2) in that she thought she obtained 1,3,5,7-tetrakis(3′/4′-bromophenyl)adamantane (8)—a mixture of isomers of 1,3,5,7-tetrakis(bromophenyl)adamantane, containing the combination of p- and m-bromophenyl groups attached to adamantane core (see below), and she considered the goal of her work not fulfilled. As support for this she writes: “The lack of regioselection during arylation discouraged us from attempting further Friedel-Crafts reactions on adamantane and lead to further study of the derivatization of the easily formed 1,3,5,7-tetraphenyladamantane” (6). To prepare single p-isomer—1,3,5,7-tetrakis[4′-(phenylethynyl)phenyl)]adamantane (7) she designed a “detour procedure”, as follows:
1,3,5,7-tetraphenyladamantane (6)→1,3,5,7-tetrakis(4′-iodophenyl)adamantane (7)→1,3,5,7-tetrakis[4′-(phenylethynyl)phenyl]adamantane (8)

Reichert successively realized this sequence, and isolated the single p-isomer (8), but the solubility of this compound turned out to be so low, that she was not able to obtain ¹³C NMR spectra of this compound. Reichert observes: “Compound 3 [(8)] was found to be soluble enough in chloroform that a ¹H NMR spectrum could be obtained. However, acquisition times were found impractical for obtaining a solution ¹³C NMR spectrum. Solid-state NMR was used to identify the product.” Reichert, Diss.(supra). And to confirm these results, Reichert's compound was tested with several standard organic solvents and was found to be essentially insoluble in every one of the tested organic solvents.

So, in other words, Reichert prepared what she thought was 1,3,5,7-tetrakis(3′/4′-bromophenyl)adamantane (3), but did not continue in this direction, because this product was not a single isomer with definite structure. Instead she prepared single isomer of 1,3,5,7-tetrakis(4′-iodophenyl)adamantane (7), and transformed it into single isomer of 1,3,5,7-tetrakis[4′-(phenylethynyl)phenyl]adamantane (8), which turned out to be insoluble, and because of that not useful.

Our Commonly Assigned Pending Patent Application PCT/US01/22204 Filed Oct. 17, 2001:

We repeated the Reichert reaction of 1,3,5,7-tetrabromoadamantane with bromobenzene numerous times and our analysis of the reaction product of 1,3,5,7-tetrabromoadamantane with bromobenzene showed that it was not 1,3,5,7-tetrakis(3′/4′-bromophenyl)adamantane (3) (as Reichert suggested), but a mixture of 1,3,5,7-tetrakis(3′/4′-bromophenyl)adamantane (3) with approximately equal quantity of 1-phenyl-3,5,7-tris(3′/4′-bromophenyl)adamantane (4). This conclusion was confirmed by LC-MS study and elemental analysis.

We were able to find the cause of such reaction course. Bromobenzene is known to disproportionate essentially in the conditions of Friedel-Crafts reaction (G. A. Olah, W. S. Tolgyesi, R. E. A. Dear. J. Org. Chem., 27, 3441-3449 (1962)):
2PhBr→PhH+Br₂Φ
When benzene concentration in the reaction mixture increases, it begins to replace bromine in (1) [or bromophenyl in (3)]; benzene proportion is so high, that fast established equilibria leads to approx. equal quantities of (3) and (4).

Therefore, Reichert did not obtain (as she thought) 1,3,5,7-tetrakis(3′/4′-bromophenyl)adamantane (3); instead, she had approx. 1:1 mixture of 1,3,5,7-tetrakis(3′/4′-bromophenyl)adamantane (3) with 1-phenyl-3,5,7-tris(3′/4′-bromophenyl)adamantane (4).

To shift equilibria toward 1,3,5,7-tetrakis(3′/4′-bromophenyl)adamantane (3) side, we treated the solid reaction product of 1,3,5,7-tetrabromoadamantane with bromobenzene [1:1 mixture of 1,3,5,7-tetrakis(3′/4′-bromophenyl)adamantane (3) and 1-phenyl-3,5,7-tris(3′/4′-bromophenyl)adamantane (4)] by a new portion of bromobenzene in presence of aluminum bromide. It turned out that pure bromobenzene immediately replaced phenyl group in 1-phenyl-3,5,7-tris(3′/4′-bromophenyl)adamantane (4), so the product in solution in 30 seconds contained approximately 90-95% 1,3,5,7-tetrakis(3′/4′-bromophenyl)adamantane (3). This situation was observed for approximately 5-10 min at room temperature, after which slowly increasing concentration of benzene led to an increase of l-phenyl-3,5,7-tris(3′/4′-bromophenyl)adamantane (4) concentration, and in several hours equilibria was re-established with approximately equal concentration of 1,3,5,7-tetrakis(3′/4′-bromophenyl)adamantane (3) and 1-phenyl-3,5,7-tris(3′/4′-bromophenyl)adamantane (4).

Therefore, 1,3,5,7-tetrakis(3′/4′-bromophenyl)adamantane (3) (that Reichert thought she synthesized) can be prepared by second treatment of the solid reaction product of 1,3,5,7-tetrabromoadamantane with bromobenzene in presence of aluminum bromide.

1,3,5,7-tetrakis(3′/4′-bromophenyl)adamantane (3) subjected to Heck reaction with phenylacetylene gave a novel mixture of 95-97 weight percent of 1,3,5,7-tetra[3′/4′-(phenylethynyl)phenyl]adamantane (5) (A mixture of p- and m-isomers formed. Five isomers formed including (1) para, para, para, para-; (2) para, para, para, meta-; (3) para, para, meta, meta-; (4) para, meta, meta, meta-; and (5) meta, meta, meta, meta-. Trace o-isomer may also be present.) and 3-5 weight percent of 1,3/4-bis{1′,3′,5′-tris[3″/4″-phenylethynyl)phenyl]adamantyl-7′-yl}benzene (14 isomers formed) which was confirmed by LC-MS, GPC, NMR, and HPLC. This mixture was very soluble in toluene, xylenes, cyclohexanone, anisole, propylene glycol methyl ether acetate, mesitylene, cyclohexylacetate, etc. For example, its solubility in cyclohexanone was approximately 20%. This property enables it to be spin coated, which ensures practical use of this material, especially and in contemplated embodiments, in the field of layered materials and semiconductors.

Although the nomenclature as used herein may not necessarily adhere to strict IUPAC standards, it is widely used and understood by those skilled in the art.

### Inventive Example 1

### Dynthesis of 1,3,5,7-Tetrabromoadamantane (TBA)

1,3,5,7-Tetrabromoadamantane synthesis started from commercially available adamantane and followed the synthetic procedures as described in G. P. Sollott and E. E. Gilbert, J. Org. Chem., 45, 5405-5408 (1980), B. Schartel, V. Sttimpflin, J. Wendling, J. H. Wendorff, W. Heitz, and R. Neuhaus, Colloid Polym. Sci., 274, 911-919 (1996), or A. P. Khardin, I. A. Novakov, and S. S. Radchenko, Zh. Org. Chem., 9, 435 (1972). Quantities of up to 150 g per batch were routinely synthesized.

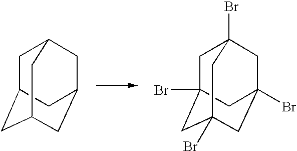

### Inventive Example 2

### Synthesis of Mixture of 1,3,5,7-Tetrakis(3′/4′-bromophenyl)adamantane (TBPA); 1,3,5-tris(3′/4′-bromophenyl)-7-phenyladamantane (TBPPA); 1,3-bis(3′/4′-bromophenyl)-5,7-diphenyladamantane (BBPDPA): and at least 1,3/4-bis[1′,3′,5′-TRIS(3″/4″-bromophenyl)adamant-7″-yl]benzene (BTBPAB)

In a first step, TBA from Inventive Example 1 was reacted with bromobenzene to yield supposedly 1,3,5,7-tetrakis(3/4-bromophenyl)adamantane (TBPA) as described in Macromolecules, 27, 7015-7023 (1994) (supra). HPLC-MS analysis showed that of the total reaction product the percentage of the desired TBPA present was approximately 50%, accompanied by 40% of the tribrominated tetraphenyladamantane, and about 10% of the dibrominated tetraphenyladamantane.

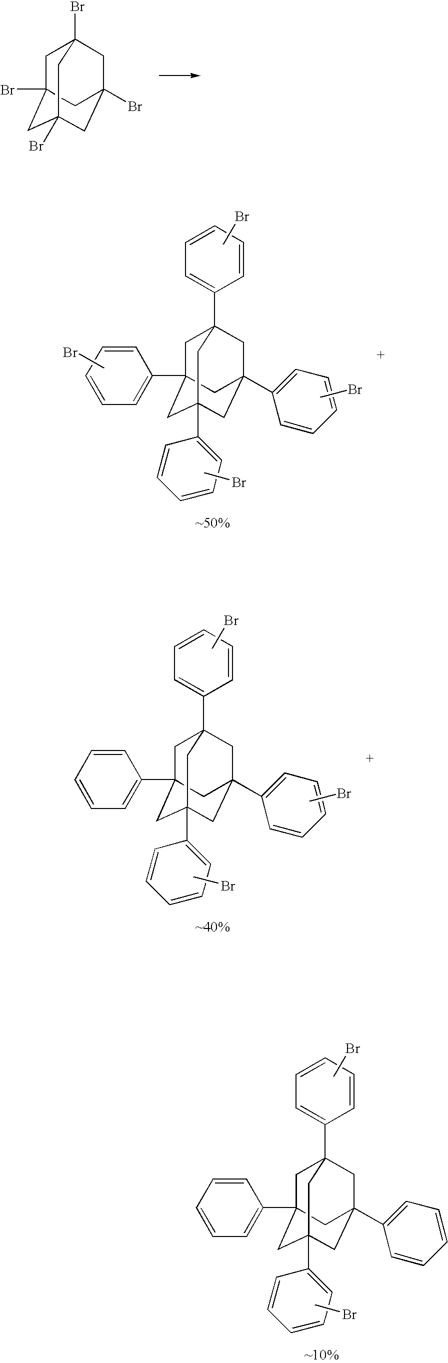

Specifically, the experimental-procedure for Step 2 above follows:

A dry 5 L 3-neck round bottom flask, water condenser, magnetic stir-bar, heating mantle, thermocouple, thermal controller unit, and N₂ inlet-outlet to 30% KOH solution were assembled. The flask was purged with N₂ for 10 min: 2 L (62% v/v from total volume) of brormobenzene were poured into the flask and the stir-bar was activated. TBA (160.00±0.30 g) was added and the funnel was rinsed with IL (31% v/v from total volume) of bromobenzene. An HPLC sample of starting material was taken and compared with standard HPLC chromatogram. Aluminum bromide (32.25±0.30g) was added to the solution and the funnel was rinsed with 220 mL (7% v/v from total volume) of bromobenzene. Solution at this point was dark purple with no precipitation visible. The reaction mixture was stirred for 1 hour at room temperature. After 1 hour, the reaction mixture temperature was raised to 40° C. After temperature reached 40° C., the reaction mixture was stirred for 3 hours. An HPLC sample was taken at time 1±3.0, respectively, at 40° C. The reaction was over when no traces of TBA were seen on HPLC chromatogram. When the reaction was over, the dark reaction mixture was poured into a 20 L reactor containing 7 L (217% v/v relative to the total volume of bromobenzene) deionized water, 2 L (62% v/v relative to the total volume of bromobenzene) ice, and 300 mL (37%) HCl (9% v/v relative to the total volume of bromobenzene). The reaction mixture was stirred vigorously using an overhead-stirrer for 1 hr±10 min.

The organic layer was transferred to a separatory chamber and washed twice with 700 mL (22% v/v relative to the total volume of bromobenzene) portions of de-ionized water. The washed organic layer was placed in a 4 L separatory funnel and added, as a slow stream, to 16 L (5×times to the total volume of bromobenzene) methanol, in a 30 L reactor placed under an overhead-stirrer, to precipitate a solid during 25 min±5 min. After the addition was complete, the methanol suspension was agitated vigorously for 1 hr±10 min. The methanol suspension was filtered by suction through a Buchner funnel (185 mm). The solid was washed on filter cake with three portions of 600 mL (19% v/v relative to the total volume of bromobenzene) methanol. The solid was suctioned dry for 30 min.

The resulting pinkish powder was emptied into a crystallizer dish using a spatula and placed in a vacuum-oven to dry overnight and then weighed after drying. The powder was re-dried in the vacuum-oven for 2 additional hours until the weight change was <1% and re-weighed. After solid was dried, the final weight was recorded and the yield was calculated. The product was as described above of approximately 50% TBPA, 40% tribrominated tetraphenyladamantane, and 10% dibrominated tetraphenyladamantane. The yield was 176.75 grams. 3-5 weight percent of BTBPAB formed.

### Inventive Example 3

### Synthesis of Mixture of 1,3,5,7-Tetrakis(3′/4′-bromophenyl)adamantane (TBPA) and 1,3/4-bis[1′,3′,5′-tris(3″/4″-bromophenyl)adamant-7′-yl]benzene (BTBPAB)

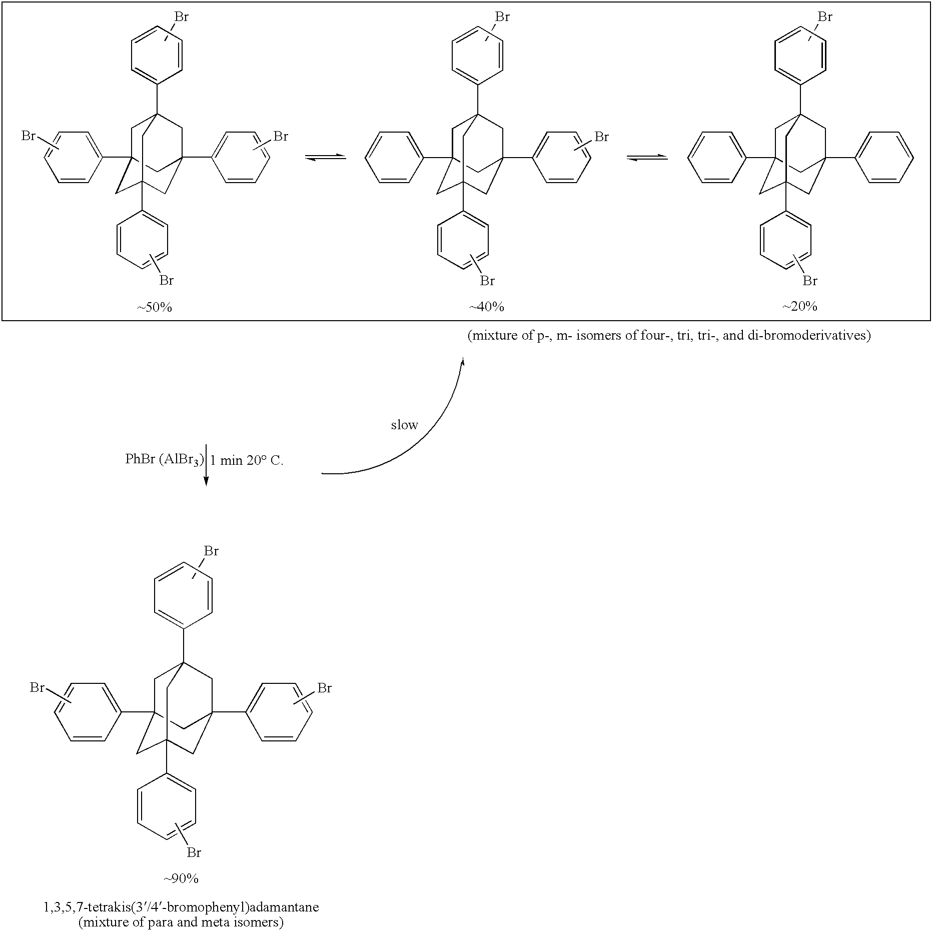

Unexpectedly, however, when the preceding product mixture from Inventive Example 2 was subjected to fresh reagent and catalyst (bromobenzene and AlCl₃, 1 min at 20° C.), the TBPA proportion of the mixture of the tetrabrominated, tribrominated, and dibrominated monomers increased from about 50% to approximately 90-95%. 3-5 weight percent of BTBPAB remained. We were so surprised by this result that we repeated it several times to confirm and this resulted in a novel process for converting the preceding mixture to a thermosetting component (a), as described below and set forth above.

Specifically, the experimental procedure for Inventive Example 3 above follows. The equipment used was the same as that of Inventive Example 2 above.

S The corresponding amounts of bromobenzene and aluminum bromide needed were calculated based on the yield of the TBPA synthesized in the above/conventional synthesis. The appropriate amount (80% v/v from the total volume) of bromobenzene was poured into the flask and the stir-bar was activated. The fall amount of TBPA from the Step 2 synthesis above was added and the funnel was rinsed with appropriate amount (10% v/v from the total volume) of bromobenzene. An HPLC sample of starting material was taken and compared with standard HPLC chromatogram. The full amount of aluminum bromide was added to the solution and the funnel was rinsed with remainder (10% from the total volume) of bromobenzene. The solution at this point was dark purple with no precipitation visible. The reaction mixture was stirred for 17 min at room temperature. An RPLC sample was taken after 5 min and after 17 min. The reaction was over when the group of peaks corresponding to TBPA was dominant in the HPLC chromatogram. When the reaction was over, the dark reaction mixture was poured into a 20 L reactor containing 7 L (217% v/v relative to the total volume of bromobenzene) deionized water, 2 L (62% v/v relative to the total volume of bromobenzene) ice, and 300 mL (37%) HCl (9% v/v relative to the total volume of bromobenzene), and stirred vigorously using an overhead-stirrer for 1 hr±10 min.

The organic layer was transferred to a separatory funnel and washed twice with 700 mL (22% v/v to the total volume of bromobenzene) portions of deionized water and 3 times with 700 mL (22% v/v relative to the total volume of bromobenzene) portions of saturated NaCl solution. The washed organic layer was placed in a 4 L separatory funnel and added, as a slow stream, to the appropriate amount (5×times to the total volume of bromobenzene) methanol, in a 30 L reactor placed under an overhead-stirrer, to precipitate a solid for 25 min±5 min. After addition was complete, the methanol suspension was agitated vigorously for 1 hr±10 min. The methanol suspension was filtered by suction through a Buchner funnel (185 mm). The solid was washed on filter cake with three portions of 600 mL (19% v/v relative to the total amount of bromobenzene) methanol. The solid was suctioned dry for 30 min.

The resulting pinkish powder was emptied into a crystallizer dish using a spatula, placed in an oven to dry overnight, weighed after drying, and re-dried in the vacuum-oven for 2 additional hours, until the weight change was <1%, and re-weighed. After the solid was dried, the final weight was recorded and the yield was calculated. The yield was 85%.

### Inventive Example 4

### Synthesis of Mixture of 1,3,5,7-Tetrakis(3′/4′-bromophenyl)adamantane (TBPA); 1,3/4-bis[1′,3′,5′-tris(3″/4″-bromophenyl)adamant-7″-yl]benzene (BTBPAB); and at least 1,3-bis(3′/4′-bromophenyl)-5,7-bis{3″/4″-[1′″,3′″5′″-tris(3″″/4″″-bromophenyl)adamant-7′″-yl]phenyl}adamantane

A first reaction vessel was loaded with adamantane (200 grams), bromobenzene (1550 milliliters), and aluminum trichloride (50 grams). The reaction mixture was heated to 40° C. by a thermostatted water bath. Tert-butyl bromide (1206 grams) was added slowly over a period of 4-6 hours to the reaction mixture. The reaction mixture at 40° C. was stirred overnight.

A second reaction vessel was loaded with 1000 milliliters of aqueous hydrogen chloride (5% w/w). The contents of the first reaction vessel were gradually discharged into the second reaction vessel while maintaining the reaction mixture at 25-35° C. by an external ice bath. An organic phase (dark brown lower phase) was separated and washed with water (1000 milliliters). About 1700 milliliters of the organic phase remained.

A third reaction vessel was loaded with 20.4 liters of petroleum ether (mainly isooctane with a boiling range of 80° C.-110° C.). The contents of the second reaction vessel were slowly added over a period of one hour to the third reaction vessel. The resulting mixture was stirred for at least one hour. The precipitate was filtered off and the filter cake was washed twice with 300 milliliters per wash of the aforementioned petroleum ether. The washed filter cake was dried overnight at 45° C. at 40 mbar. The product yield was 407 grams dry weight.

Analytical techniques including GPC, HPLC, and NMR were used to identify the product. GPC analysis showed: 1,3,5,7-tetrakis(3′/4′-bromophenyl)adamantane had a peak molecular weight of 430; 1,3/4-bis[1′,3′,5′-tris(3″/4″-bromophenyl)adamant-7′-yl]benzene had a peak molecular weight of 820; 1,3-bis{3′/4′-[1″,3″,5″-tris(3′″/4′″-bromophenyl)adamant-7″-yl]phenyl}-5,7-bis(3″″/4″″-bromophenyl)adamantane had a peak molecular weight of about 1150 (shoulder).

### Inventive Example 5

### Synthesis of Mixture of 1,3,5,7-Tetrakis(3′/4′-bromophenyl)adamantane (TBPA); 1,3/4-bis[1′,3′,5′-tris(3″/4″-bromophenyl)adamant-7″-yl]benzene (BTBPAB); and at least 1,3-bis(3′/4′-bromophenyl)-5,7-bis{3″/4″-[1′″,3′″,5′″-tris(3″″/4″″-bromophenyl)adamant-7″-yl]phenyl}adamantane

A first reaction vessel was loaded with 1,4-dibromobenzene (587.4 grams) and aluminum trichloride (27.7 grams). This reaction mixture was heated to 90° C. by a thermostatted water bath and maintained at this temperature for one hour without stirring and for an additional one hour with stirring. The reaction mixture was cooled down to 50° C. Adamantane (113.1 grams) was added to the cooled reaction mixture. Over a period of four hours, t-butyl-bromobenzene (796.3 grams) was-added to the reaction mixture. The reaction mixture was stirred for an additional 12 hours.

A second reaction vessel was loaded with HCl (566 milliliters, 10% aqueous w/w). The contents of the first reaction vessel at 50° C. were discharged into the second reaction vessel while maintaining the mixture at 25-35° C. by an external ice bath. The reaction mass was a light brown suspension. The organic phase was a dark brown lower phase and separated from the reaction mixture. The separated organic phase was washed with water (380 milliliters). After this washing, about 800 milliliters of organic phase remained.

A third reaction vessel was loaded with heptane (5600 milliliters). Slowly over a period of one hour, the contents of the second reaction vessel were added to the third reaction vessel. The suspension was stirred for at least four hours and the precipitate was filtered off. The filter cake was washed twice with 300 milliliters per wash of heptane. The IE2 Step (a) Product yield was 526.9 grams (wet) and 470.1 grams (dry).

Analytical techniques including GPC, HPLC, and NMR were used to identify the product. GPC analysis showed: 1,3,5,7-tetrakis(3′/4′-bromophenyl)adamantane had a peak molecular weight of about 430; 1,3/4-bis[1′,3′,5′-tris(3″/4″-bromophenyl)adamant-7′-yl]benzene had a peak molecular weight of about 820; 1,3-bis{3′/4′-[1″,3″,5″-tris(3′″/4′″-bromophenyl)adamant-7″-yl]phenyl}-5,7-bis(3″″/4″″-bromophenyl)adamantane had a peak molecular weight of about 1150 (shoulder).

### Inventive Example 6

### Synthesis of Mixture of 1,3-bis{3″/4″-[1″-phenyl-3″,5″-bis(3′″/4′″-(phenylethynyl)phenyl)adamant-7″-yl]phenylethynyl}benzene; 1,3-bis{3′/4′-[1″,3″,5″-TRIS(3′″/4′″-(phenylethynyl)phenyl)adamant-7″-yl]phenylethynyl}benzene; and at least 1,3-bis[3^{[1]}/4^{[1]}-(phenylethynyl)phenyl]-5,7-bis{3^{[2]}/4^{[2]}-[3^{[3]}-(3^{[4]}/4^{[4]}-(1^{[1]},3^{[5]},5^{[5]}-tris(3^{[6]}/4^{[6]}-(phenylethenyl)phenyl)adamant-7^{[5]}-yl)phenylethynyl)phenylethenyl]phenyl}adamantane

To a dry 500 mL 3-necked round bottom flask equipped with a water condenser, an overhead stirrer, a thermocouple, a nitrogen inlet-outlet and a heating mantle were added 20.00 g (26.45 mmol) of TBPA from Inventive Example 2, 1.4850 g (2.1158 mmol) of dichlorobis(triphenylphosphine)palladium (II), 1.1099 g (4.2316 mmol) of triphenylphosphine, 0.4029 g (2.1158 mmol) of copper (I) iodide, and 119 mL of triethylamine. The mixture was stirred at room temperature for 5 minutes and then heated to 80° C. To this mixture were added 0.8341 g (6.6118 mmol) of m-diethynylbenzene in 5 mL of triethylamine through an additional funnel dropwise. The reaction mixture was heated at 80° C. for 2 hours and then 21.6106 g (211.579 mmol) of phenylacetylene in 5 mL of triethylamine through an additional funnel were added dropwise. The reaction mixture was heated at 80° C. for additional 4 hours. The reaction mixture was cooled to room temperature and transferred to a 1 L, 3 neck flask equipped with a condenser, a mechanical stirrer and a nitrogen inlet-outlet and 100 ml of toluene was added. The solution was then neutralized with 6N HCl. The resulting water was removed. The toluene solution was then stirred with 100 mL of 6N HCl at 60° C. for 30 min. The mixture was filtered through celite®. The aqueous solution was then removed. The HCl extraction was repeated for two more times. The toluene solution was then washed with 100 mL of deionized water twice. The solution was stirred with 100 mL of 0.1 M of N-acetyl-cysteine in ammonia solution at 60° C. for 30 min. The aqueous solution was then removed. The ammonia extraction was repeated for five more times. The toluene was then removed by rotary evaporator and the resulting solid was dried under vacuum overnight to yield 18.70 g (84%) of reddish Solid.

### Inventive Example 7

### Synthesis of Mixture of 1,3-bis{3′/4′-[1″,3″,5″-tris(3′″/4′″-(phenylethynyl)phenyl)adamant-7″-yl]phenylethynyl}benzene, and at least 1,3-bis[3^{[1]}/4^{[1]}-(phenylethynyl)phenyl]-5,7-bis{3^{[2}/4^{[2]}-[3^{[3]}-(3^{[4]}/4^{[4]}-(1^{[5]},3^{[5]},5^{[5]}-tris(3^{[6]}/4^{[6]}-(phenylethynyl)phenyl)adamant-7^{[5]}-yl)phenylethynyl)phenylethynyl]phenyl}adamantane

Synthesis of this product is similar to that in Inventive Example 6 except TBPA from Inventive Example 3 was used instead of TBPA from Inventive Example 2.

### Inventive Example 8

### Synthesis of Mixture of 1,3-bis{3′/4′-[1″,3″,5″-tris(3′″/4′″-(phenylethynyl)phenyl)adamant-7″-yl]phenylethynyl}benzene; 1,3-bis[3^{[1]}/4^{[1]}-(phenylethynyl)phenyl]-5,7-bis{3^{[2]}/4^{[2]}-[3^{[3]}-(3^{[4]}/4^{[4]}-(1^{[5]},3^{[5]},5^{[5]}-tris(3^{[6]}4^{[6]}-(phenylethynyl)phenyl)adamant-7^{[5]}-yl)phenylethynyl)phenylethynyl]phenyl}adamantane; and at least 1,3-bis(3^{[1]}/4^{[1]}-(phenylethynyl)phenyl)-5-{3^{[2]}/4^{[2]}-[1^{[3]},3^{[3]},5^{[3]}-tris(3^{[4]}/4^{[4]}-(phenylethynyl)phenyl)adamant-7^{[3]}-yl]phenyl}-7-{3^{[5]}/4^{[5]}-[3^{[6]}-(3^{[7]}/4^{[7]}-(1^{[8]},3^{[8]},5^{[8]}-tris(3^{[9]}/4^{[9]}-(phenylethynyl)phenyl)adamant-7^{[8]}-yl)phenylethynyl)phenylethynyl]phenyl}adamantane

Synthesis of this product is similar to that in Inventive Example 6, except TBPA from Inventive Example 4 was used instead of TBPA from Inventive Example 2.

### Inventive Example 9

### Synthesis of 1,3-bis{3′/4′-[1″,3″,5″-tris(3′″/4′″-(phenylethynyl)phenyl)adamant-7″-yl]phenylethynyl}benzene; 1,3-bis[3^{[1]}/4^{[1]}-(phenylethynyl)phenyl]-5,7-bis{3^{[2]}/4^{[2]}-[3^{[3]}-(3^{[4]}/4^{[4]}-(1^{[5]},3^{[5]},5^{[5]}-tris(3^{[6]}/4^{[6]}-(phenylethynyl)phenyl)adamant-7^{[5]}-yl)phenylethynyl)phenylethynyl]phenyl}adamantane; and at least 1,3-bis(3^{[1]}/4^{[1]}-(phenylethynyl)phenyl)-5-{3^{[2]}/4^{[2]}-]1^{[3]},3^{[3]},5^{[3]}-tris(3^{[4]}/4^{[4]}-(phenylethynyl)phenyl)adamant-7^{[3]}-yl]phenyl}-7-{3^{[5}/4^{[5]}-[3^{[6]}-(3^{[7]}/4^{[7]}-(1^{[8]},3^{[8]},5^{[8]}-tris(3^{[9]}/4^{[9]}-(phenylethynyl)phenyl)adamant-7^{[8]}-yl)phenylethynyl)phenylethynyl]phenyl}adamantane with Reduced Amount of Oligomer Compared to Inventive Example 8

To a dry 250 mL 3-necked round bottom flask equipped with a water condenser, an overhead stirrer, a thermocouple, a nitrogen inlet-outlet and a heating mantle were added 10.00 g (11.29 mmol) of TBPA from Inventive Example 4, 0.6335 g (0.9026 mmol) of dichlorobis(triphenylphosphine)palladium (II), 0.4735 g (1.8053 mmol) of triphenylphosphine, 0.1719 g (0.9026 mmol) of copper (I) iodide, and 50 mL of triethylamine. The mixture was stirred at room temperature for 5 minutes and then heated to 80° C. To this mixture were added 2.3049 g (22.566 mmol) of phenylacetylene in 2 mL of triethylamine through an additional funnel dropwise. The reaction mixture was heated at 80° C. for 4 hours. To this mixture were then added 0.7117 g (5.6415 mmol) of m-diethynylbenzene in 2 mL of triethylamine through an additional funnel dropwise. The reaction mixture was heated at 80° C. for 2 hours and then 6.9146 g (67.6975 mmol) of phenylacetylene in 6 mL of triethylamine through an additional funnel were added dropwise. The reaction mixture was heated at 80° C. for additional 4 hours. The purification of the reaction mixture is similar to Inventive Example 7.

### Inventive Example 10

### Synthesis of 1,3-bis{3′/4′-[1″,3″,5″-tris(3′″/4′″-(phenylethynyl)phenyl)adamant-7″-yl]phenylethynyl}benzene; 1,3-bis[3^{[1]}/4^{[1]}-(phenylethynyl)phenyl]-5,7-bis{3^{[2]}/4^{[2]}-[3^{[3]}-(3^{[4]}/4^{[4]}-(1^{[5]},3^{[5]},5^{[5]}-tris(3^{[6]}/4^{[6]}-(phenylethynyl)phenyl)adamant-7^{[5]}-yl)phenylethynyl)phenylethynyl]phenyl}adamantane; and at least 1,3-bis(3^{[1]}/4^{[1]}-(phenylethynyl)phenyl)-5-{3^{[2]}/4^{[2]}-[1^{[3]},3^{[3]},5^{[3]}-tris(3^{[4]}/4^{[4]}-(phenylethynyl)phenyl)adamant-7^{[3]}-yl[phenyl}-7-{3^{[5]}/4^{[5]}-[3^{6]}-(3^{[7]}/4^{[7]}-(1^{[8]},3^{[8]},5^{[8]}-tris(3^{[9]}/4^{[9]}-(phenylethynyl)phenyl)adamant-7^{[8]}-yl)phenylethynyl)phenylethynyl]phenyl}adamantane with more reduced amount of oligomer compared to Inventive Examples 8 and 9

To a dry 250 mL 3-necked round bottom flask equipped with a water condenser, an overhead stirrer, a thermocouple, a nitrogen inlet-outlet and a heating mantle were added 10.00 g (11.29 mmol) of TBPA from Inventive Example 4, 0.6335 g (0.9026 mmol) of dichlorobis(triphenylphosphine)palladium (II), 0.4735 g (1.8053 mmol) of triphenylphosphine, 0.1719 g (0.9026 mmol) of copper (I) iodide, and 48 mL of triethylamine. The mixture was stirred at room temperature for 5 minutes and then heated to 80° C. To this mixture were added 4.0335 g (39.490 mmol) of phenylacetylene in 2 mL of triethylamine through an additional funnel dropwise. The reaction mixture was heated at 80° C. for 4 hours. To this mixture were then added 0.7117 g (5.6415 mmol) of m-diethynylbenzene in 2 mL of triethylamine through an additional funnel dropwise. The reaction mixture was heated at 80° C. for 2 hours and then 4.6098 g (45.1317 mmol) of phenylacetylene in 6 mL of triethylamine through an additional funnel were added dropwise. The reaction mixture was heated at 80° C. for additional 4 hours. The purification of the reaction mixture is similar to example in 7.

### Inventive Example 11

### Synthesis of 1,3-bis{3′/4′-[1″,3″,5″-tris(3′″/4′″-(phenylethynyl)phenyl)adamant-7′-yl]phenylethynyl}benzene: 1,4-bis{3′/4′-[1″,3″,5″-tris(3′″/4′″-(phenylethynyl)phenyl)adamant-7″-yl]phenylethynyl}benzene; and at least 1,3-bis[3^{[1]}/4^{[1]}-(phenylethynyl)phenyl]-5-{3^{[2]}/4^{[2]}-[3^{[3]}-(3^{[4]}/4^{[4]}-(1^{[5]},3^{[5]},5^{[5]}-tris(3^{[6]}/4^{[6]}-(phenylethynyl)phenyl)adamant-7^{[5]}-yl)phenylethynyl)phenylethynyl]phenyl}-7-{3^{[7]}/4^{[7]}-[4^{[8]}-(3^{[9]}/4^{[9]}-(1^{[10]},3^{[10]},5^{[10]}-tris(3^{[11]}/4^{[11]}-(phenylethynyl)phenyl)adamant-7^{[10]}-yl)phenylethynyl)phenylethynyl]phenyl}adamantane

To a dry 250 mL 3-necked round bottom flask equipped with a water condenser, an overhead stirrer, a thermocouple, a nitrogen inlet-outlet and a heating mantle were added 8.2040 g (9.2565 mmol) of TBPA from Inventive Example 4 above, 0.5198 g (0.7405 mmol) of dichlorobis(triphenylphosphine)palladium (II), 0.3885 g (1.4810 mmol) of triphenylphosphine, 0.1410 g (0.7405 mmol) of copper (I) iodide, and 41 mL of triethylamine. The mixture was stirred at room temperature for 5 minutes and then heated to 80° C. To this mixture were added 0.2919 g (2.3141 mmol) of m-diethynylbenzene and 0.2919 g (2.3141 mmol) of p-diethynylbenzene in 4 mL of triethylamine through an additional funnel dropwise. The reaction mixture was heated at 80° C. for 8 hours and then 7.5637 g (74.0521 mmol) of phenylacetylene in 5 mL of triethylamine through an additional funnel were added dropwise. The reaction mixture was heated at 80° C. for additional 4 hours. The purification of the reaction mixture is similar to example in 7.

### Inventive Example 12

### Synthesis of 1,3-bis{3′/4′-[1″,3″,5″-tris(3′″/4′″-(phenylethynyl)phenyl)adamant-7″-yl]phenylethynyl}benzene: 1,4-bis3′/4′-[1″,3″,5″-tris(3′″/4′″-(phenylethynyl)phenyl)adamant-7″-yl]phenylethynyl}benzene: and at least 1,3-bis[3^{[1]}/4^{[1]}-(phenylethynyl)phenyl]-5-{3^{[2]}/4^{[2]}-[3^{[3]}-(3^{[4]}/4^{[4]}-(1^{[5]},3^{[5]},5^{[5]}-tris(3^{[6]}/4^{[6]}-(phenylethynyl)phenyl)adamant-7^{[5]}-yl)phenylethynyl)phenylethynyl]phenyl}-7-{3^{[7]}/4^{[7]}-[4^{[8]}-(3^{[9]}/4^{[9]}-(1^{[10]},3^{[10]},5^{[10]}-tris(3^{[11]}/4^{[11]}-(phenylethynyl)phenyl)adamant-7^{[10]}-yl)phenylethynyl)phenylethynyl]phenyl}adamantane with reduced amount of oligomer compared to Inventive Example 11

To a dry 250 mL 3-necked round bottom flask equipped with a water condenser, an overhead stirrer, a thermocouple, a nitrogen inlet-outlet and a heating mantle were added 11.72 g (13.22 mmol) of TBPA from Inventive Example 4, 0.7425 g (1.0579 mmol) of dichlorobis(triphenylphosphine)palladium (II), 0.5549 g (2.1158 mmol) of triphenylphosphine, 0.2015 g (1.0579 mmol) of copper (I) iodide, and 58 mL of triethylamine. The mixture was stirred at room temperature for 5 minutes and then heated to 80° C. To this mixture were added 2.7013 g (26.4472 mmol) of phenylacetylene in 3 mL of triethylamine through an additional funnel dropwise. The reaction mixture was heated at 80° C. for 4 hours. To this mixture were then added 0.4170 g (3.3059 mmol) of m-diethynylbenzene and 0.4170 g (3.3059 mmol) of p-diethynylbenzene in 6 mL of triethylamine through an additional funnel dropwise. The reaction mixture was heated at 80° C. for 8 hours and then 8.1039 g (79.3415 mmol) of phenylacetylene in 7 mL of triethylamine through an additional funnel were added dropwise. The reaction mixture was heated at 80° C. for additional 4 hours. The purification of the reaction mixture is similar to example in 7.

### Inventive Example 13

### Synthesis of 1,3-bis{3′/4′-[1″,3″,5″-tris(3′″/4′″-(phenylethynyl)phenyl)adamant-7″-yl]phenylethynyl}benzene: 1,4-bis{3′/4′-[1″,3″,5″-tris(3′″/4′″-(phenylethynyl)phenyl)adamant-7″-yl]phenylethynyl}benzene; and at least 1,3-bis[3^{[1]}/4^{[1]}-(phenylethynyl)phenyl]-5-{3^{[2]}/4^{[2]}-[3^{[3]}-(3^{[4]}/4^{[4]}-(1^{[5]},3^{[5]},5^{[5]}-tris(3^{[6]}/4^{[6]}-(phenylethynyl)phenyl)adamant-7^{[5]}-yl)phenylethynyl)phenylethynyl]phenyl}-7-{3^{[7]}/4^{[7]}-[4^{[8]}-(3^{[9]}/4^{[9]}-(1^{[10]},3^{[10]},5^{[10]}-tris(3^{[11]}/4^{[11]}-(phenylethynyl)phenyl)adamant-7^{[10]}-yl)phenylethynyl)phenylethynyl]phenyl}adamantane with more reduced amount of oligomer compared to Inventive Examples 11 AND 12

To a dry 500 mL 3-necked round bottom flask equipped with a water condenser, an overhead stirrer, a thermocouple, a nitrogen inlet-outlet and a heating mantle were added 35.16 g (39.67 mmol) of TBPA from Inventive Example 4 above, 2.2276 g (3.1737 mmol) of dichlorobis(triphenylphosphine)palladium (II), 1.6648 g (6.3473 mmol) of triphenylphosphine, 0.6044 g (3.1737 mmol) of copper (I) iodide, and 170 mL of triethylamine. The mixture was stirred at room temperature for 5 minutes and then heated to 80° C. To this mixture were added 14.1819 g (138.848 mmol) of phenylacetylene in 9 mL of triethylamine through an additional funnel dropwise. The reaction mixture was heated at 80° C. for 4 hours. To this mixture were then added 1.2511 g (9.9177 mmol) of m-diethynylbenzene and 1.2511 g (9.9177 mmol) of p-diethynylbenzene in 17 mL of triethylamine through an additional funnel dropwise. The reaction mixture was heated at 80° C. for 8 hours and then 16.2079 g (158.6831 mmol) of phenylacetylene in 21 mL of triethylamine through an additional funnel were added dropwise. The reaction mixture was heated at 80° C. for additional 4 hours. The purification of the reaction mixture is similar to Inventive Example 7.

### Inventive Example 14

To a dry 500 mL 3-necked round bottom flask equipped with a water condenser, a nitrogen inlet-outlet, a magnetic stirrer, and an oil bath were added 10 g of the inventive example 6, 100 g of xylenes and 0.67 g of polycarbosilane. The reaction mixture was heated at 145° C. for 15.5 hours. Most of the xylenes was then removed by rotary evaporator until a viscous liquid was obtained. To this flask was then added 100 g of cyclohexanone. Again most of the solvent was then removed by rotary evaporator until a viscous liquid was obtained. This process was repeated two more times to ensure that all the xylenes was exchanged to cyclohexanone. The solution was then diluted with cyclohexanone to make a 235 solid concentration solution.

| FORMULATION | | | POST BAKE | | | | POST CURE | |
|---|---|---|---|---|---|---|---|---|
| Inventive | | Concentration | | Thickness | CURE | | | Thickness |
| example | A3 Concentration | | Index | (A) | Temp | Time | Index | (A) |
| 6 | 6.7 | 20% | 1.69 | 11500 | 400 | 60 | 1.607 | 12000 |
| 6 | 6.7 | 22% | 1.68 | 14600 | 400 | 60 | 1.622 | 14900 |
| 6 | 6.7 | 23% | 1.679 | 16323 | 400 | 60 | 1.593 | 19637 |
| 7 | 6.7 | 22% | 1.695 | 13227 | 400 | 60 | 1.622 | 14030 |
| 8 | 0 | 8% | 1.673 | 2336 | 400 | 60 | 1.612 | 2436 |
| 8 | 0 | 8% | 1.660 | 2427 | 400 | 60 | 1.609 | 2446 |
| 8 | 0 | 8% | 1.668 | 2094 | 400 | 60 | 1.636 | 2138 |
| 8 | 6.7 | 8% | n/a | n/a | n/a | n/a | n/a | n/a |
| 8 | 6.7 | 8% | n/a | n/a | n/a | n/a | n/a | n/a |
| 9 | 0 | 8% | 1.672 | 2107 | 400 | 60 | 1.624 | 2159 |
| 9 | 6.7 | 8% | n/a | n/a | n/a | n/a | n/a | n/a |
| 9 | 6.7 | 8% | 1.680 | 2765 | 400 | 60 | 1.616 | 2824 |
| 10 | 6.7 | 8% | 1.659 | 2271 | 400 | 60 | 1.617 | 2252 |
| 11 | 0 | 8% | 1.673 | 2168 | 400 | 60 | 1.637 | 2206 |
| 11 | 6.7 | 8% | 1.616 | 2402 | 400 | 60 | 1.553 | 2481 |
| 12 | 6.7 | 8% | 1.654 | 2577 | 400 | 60 | 1.510 | 2742 |
| 13 | 6.7 | 12% | 1.680 | 3045 | 400 | 60 | 1.620 | 3089 |

### Inventive Example 15

To a plastic bottle equipped with a magnetic stirring bar were added 10.30 g of polyacenaphthylene, 0.30 g of polycarbosilane and 138.38 g of xylenes. The solution was stirred at room temperature for 24 hours. The solution was then transferred to a 500 mL three neck flask and 10.00 g of inventive example 6 and additional 19.8 g of xylene were added. The solution was flushed with nitrogen for 5 minutes and heated at 145° C. for 15.5 hours. Most of the xylenes was then removed by rotary evaporator until a viscous liquid was obtained. To this flask was then added 150 g of cyclohexanone. Again most of the solvent was then removed by rotary evaporator until a viscous liquid was obtained. This process was repeated two more times to ensure that all the xylenes was exchanged to cyclohexanone. The solution was then diluted with cyclohexanone to make a 23% solid concentration solution. The solution was filtered slowly at less than 20 pounds per square inch through a 0.1 μm teflon filter. The preceding step was repeated. The final composition was 23% solids with 3% weight percent polycarbosilane, 50% weight percent polyacenaphthylene, and the remainder was thermosetting component.

| | |
|---|---|
| PB | PC |

| Spin speed | Thickness | RI | Thickness | RI |
|---|---|---|---|---|
| 1400 | 14113.09 | 1.6795 | 14649 | 1.350 |
| 1000 | 7758.98 | 1.6804 | 7773.4 | 1.356 |

### Inventive Example 16

Synthesis of Acetylene Functionalized Porogen

To a 500-mL, 3 neck flask equipped with a condenser, a magnetic stirrer and a nitrogen inlet-outlet were added 0.262 g (60% dispersion in mineral oil, which corresponds to 6.54 mmol) of sodium hydride, and 60 ml of hexane. The mixture was stirred at room temperature for 5 min and upper hexane layer was decanted. To the above mixture were added 0.695 g (5.93 mmol) of 4-ethynylaniline and 144 g of TBF. The solution was stirred at room temperature for 1 hour and 15.0 g of epoxy functionalized polynorbornene was added. The reaction mixture was heated at 60° C. for 12 hours. THF was then removed by rotary evaporator and the resulting mixture was dissolve in 50 ml of toluene (solution A).

Heck Reaction

To a 500-mL, 3 neck flask equipped with a condenser, a mechanical stirrer and a nitrogen inlet-outlet were added 25.75 g (26.00 mmol) of S-TBPA, 1.461 g (2.081 mmol) of dichlorobis(triphenylphosphine)palladium(II), 1.092 g (4.162 mmol) of triphenylphosphine, 0.3963 g (2.081 mmol) of copper(]) iodide, 160 ml of triethylamine and 80 ml of toluene. The mixture was heated to 80° C. and 0.82 g (6.5 mmol) of m-diethynylbenzene was added to the reaction mixture dropwise. The reaction mixture was heated at 80° C. for 4 hours and then the above solution A was added to the reaction mixture dropwise. The reaction mixture was heated at 80° C. for 12 hours and then 21.30 g (208.1 mmol) of phenylacetylene and 30 ml of toluene were added to the reaction mixture dropwise. The solution was heated at 80° C. for 4 hours.

Work Up

The reaction mixture was cooled to room temperature and transferred to a 1 L, 3 neck flask equipped with a condenser, a mechanical stirrer and a nitrogen inlet-outlet and 100 ml of toluene was added. The solution was then neutralized with 6N HCl. The resulting water was removed. The toluene solution was then stirred with 100 mL of 6N HCl at 60° C. for 30 min. The mixture was filtered through celite®. The aqueous solution was then removed. The HCl extraction was repeated for two more times. The toluene solution was then washed with 100 mL of deionized water twice. The solution was stirred with 100 mL of 0.1 M of N-acetyl-cysteine in ammonia solution at 60° C. for 30 min. The aqueous solution was then removed. The ammonia extraction was repeated for five more times. The toluene was then removed by rotary evaporator and the resulting solid was dried under vacuum overnight.

Formulation

To a 125-mL plastic bottle were added 4.48 g of the above solid, 0.047 g of ortho-cresol novolac and 40.74 g of xylenes. The solution was stirred at room temperature for 1 hour. The solution was then filtered through a 0.1 μm teflon filter.

We claim:

1. A composition comprising at least one oligomer or polymer of Formula I 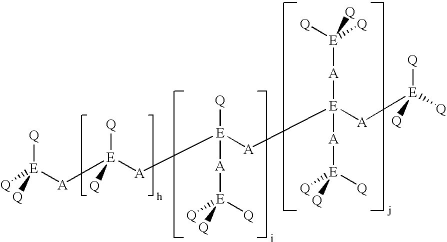
where E is a cage compound; Q is the same or different and selected from aryl, branched aryl, and substituted aryl wherein said substituents include hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl; A is substituted or unsubstituted aryl with substituted or unsubstituted arylalknyl group, wherein substituents include hydrogen, halogen, alkyl, phenyl or substituted aryl; and aryl includes phenyl, biphenyl, naphthyl, terphenyl, anthracenyl, polyphenylene, polyphenylene ether, or substituted aryl; h is from 0 to 10; i is from 0 to 10; j is from 0 to 10; and w is 0 or 1.

2. The composition of claim 1 wherein said oligomer or polymer is adamantane of Formula II 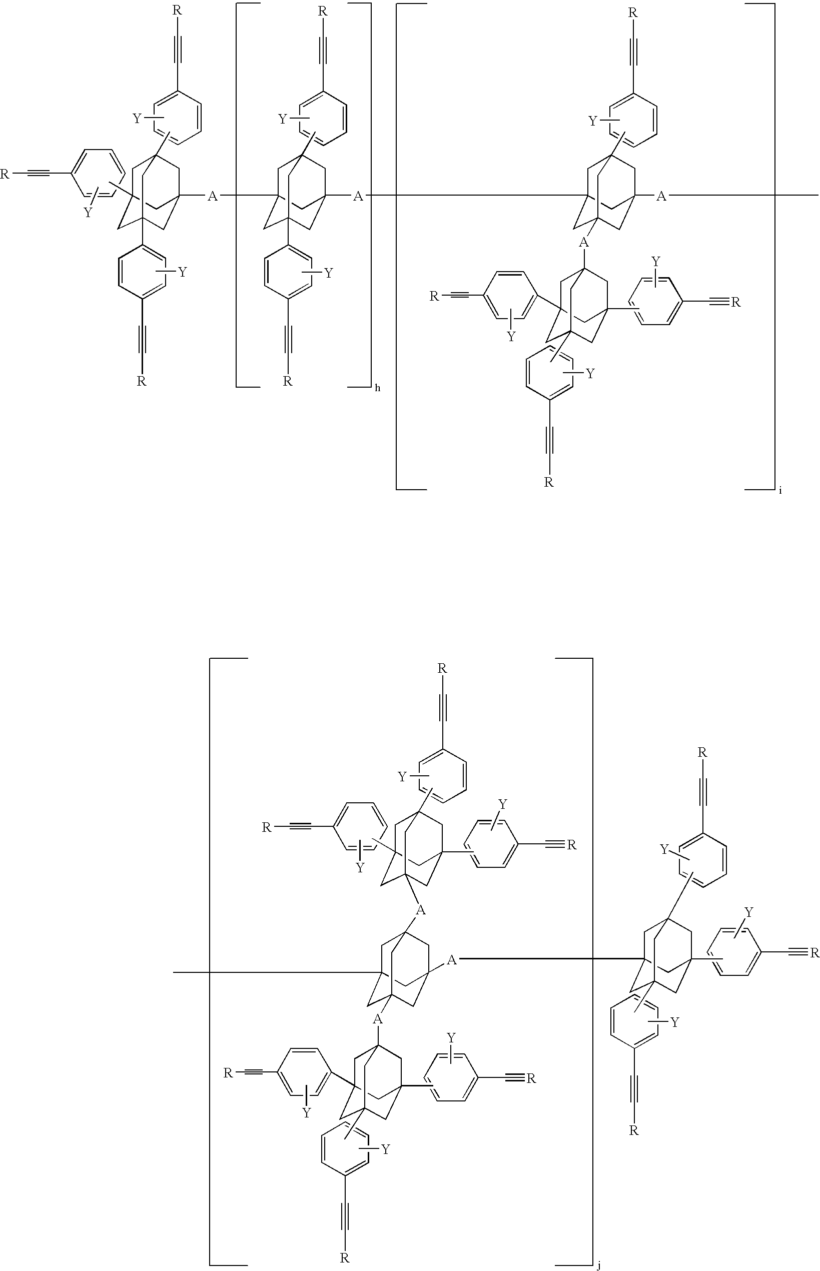
or said oligomer or polymer is diamantane monomer of Formula III 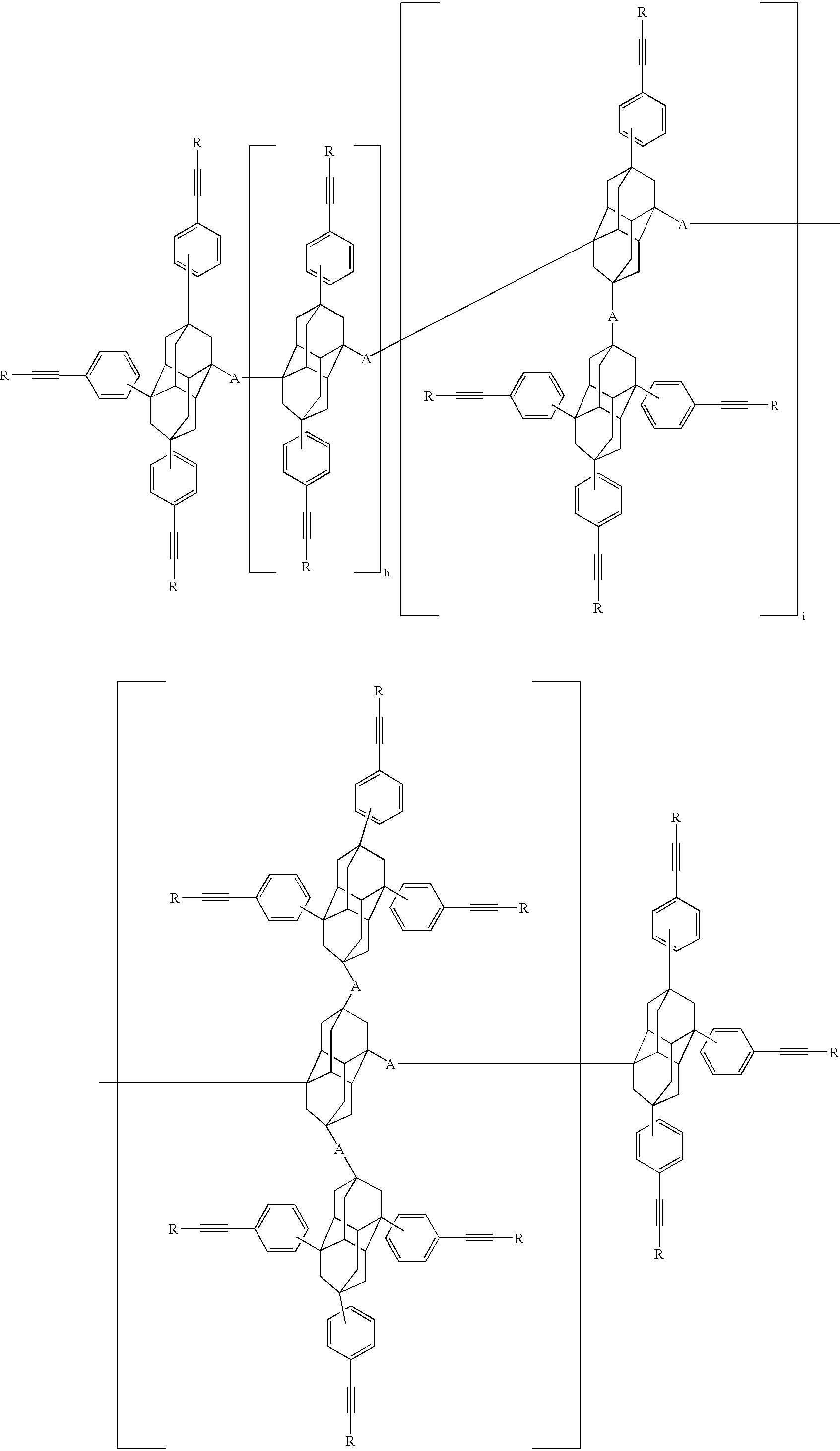
where said h is from 0 to 10; said i is from 0 to 10; said j is from 0 to 10; each of said R is the same or different and selected from hydrogen, halogen, alkyl, aryl, substituted aryl, heteroaryl, aryl ether, alkenyl, alkynyl, alkoxyl, hydroxyalkyl, hydroxyaryl, hydroxyalkenyl, hydroxyalkynyl, hydroxyl, or carboxyl; each of said Y is same or different and is selected from hydrogen, alkyl, aryl, substituted aryl, or halogen; and
each of said A is substituted or unsubstituted aryl with substituted or unsubstituted arylalkynyl group, wherein substituents include hydrogen, halogen, alkyl, phenyl or substituted aryl; and aryl includes phenyl, biphenyl, naphthyl, terphenyl, anthracenyl, polyphenylene, polyphenylene ether, or substituted aryl.

3. The composition of claim 2 wherein said R is phenyl.

4. The composition of claim 2 wherein said Y is hydrogen.

5. The composition of claim 2 wherein said adamantane oligomer or polymer is of Formula V where h is 0 or 1 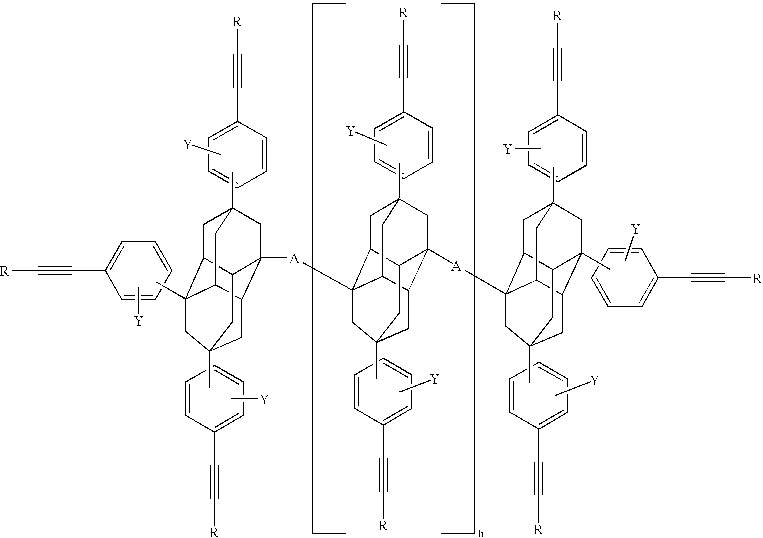
or said diamantane oligomer or polymer is of Formula VI where h is 0 or 1

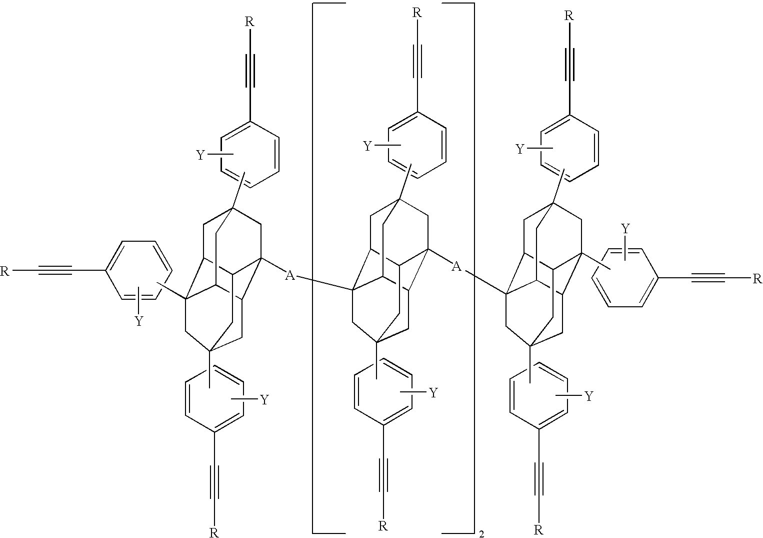

6. The composition of claim 5 wherein said adamantane oligomer or polymer is of Formula VII 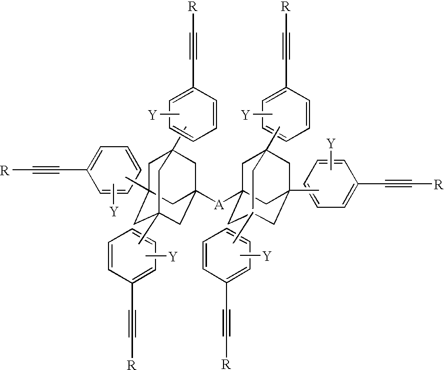
or said diamantane oligomer or polymer is of Formula VIII

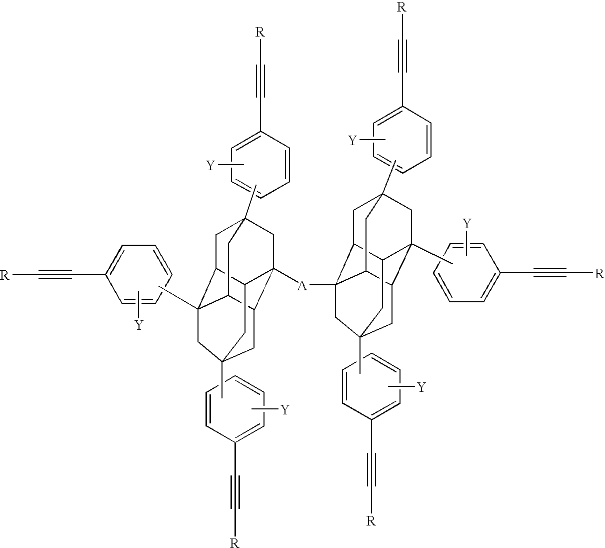

7. The composition of claim 5 wherein said adamantane oligomer or polymer is of Formula IX 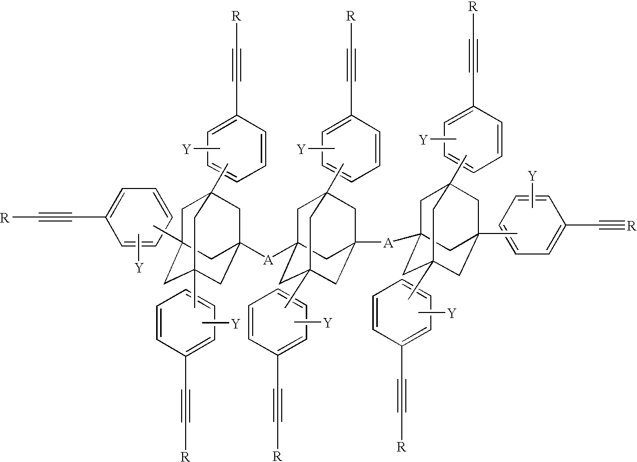
or said diamantane oligomer or polymer is of Formula X

8. The composition of claim 2 wherein said adamantane oligomer or polymer is of Formula XI 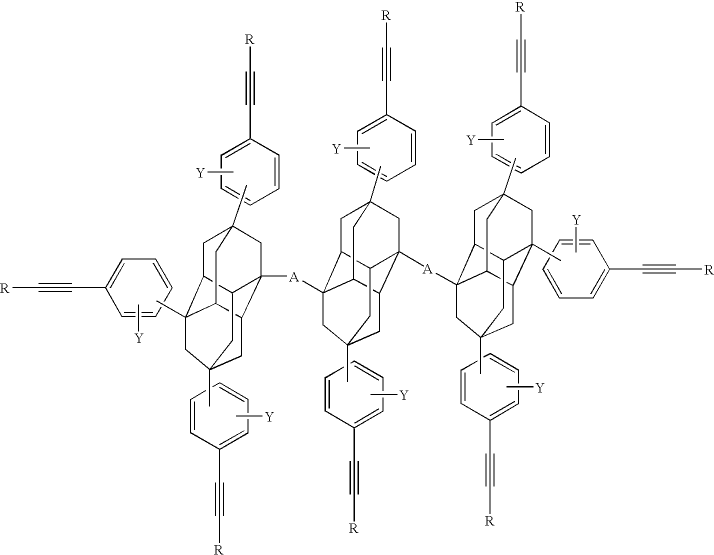 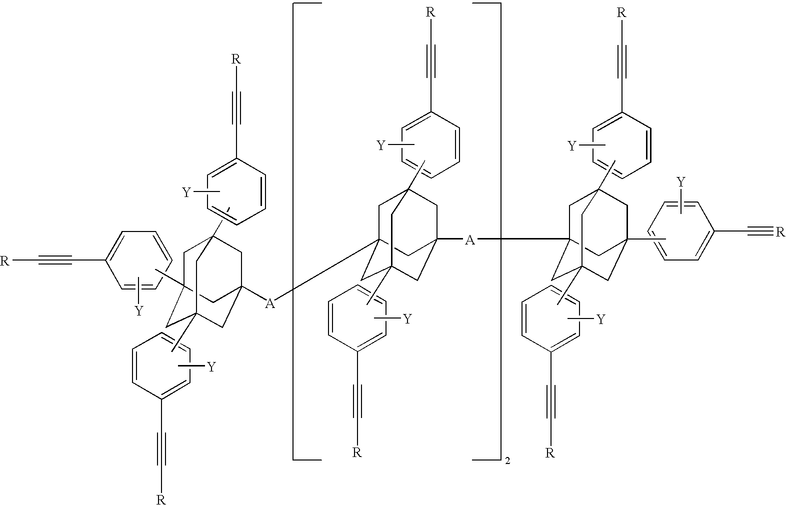
and said adamantane oligomer or polymer is of Formula XII 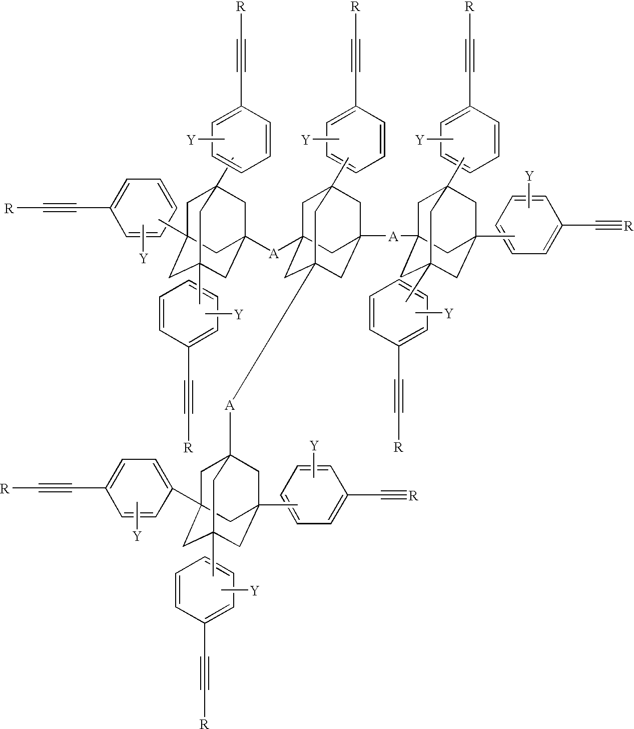
or said diamantane oligomer or polymer is of Formula XIII 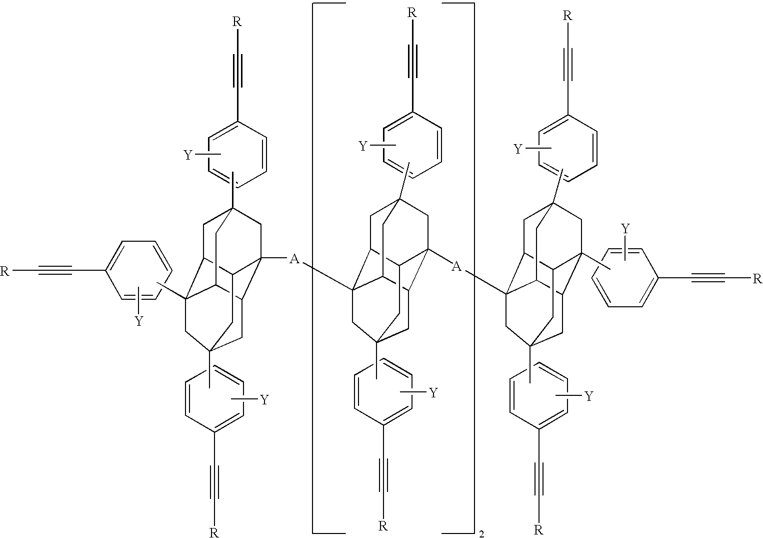
and said diamantane oligomer or polymer is of Formula XIV

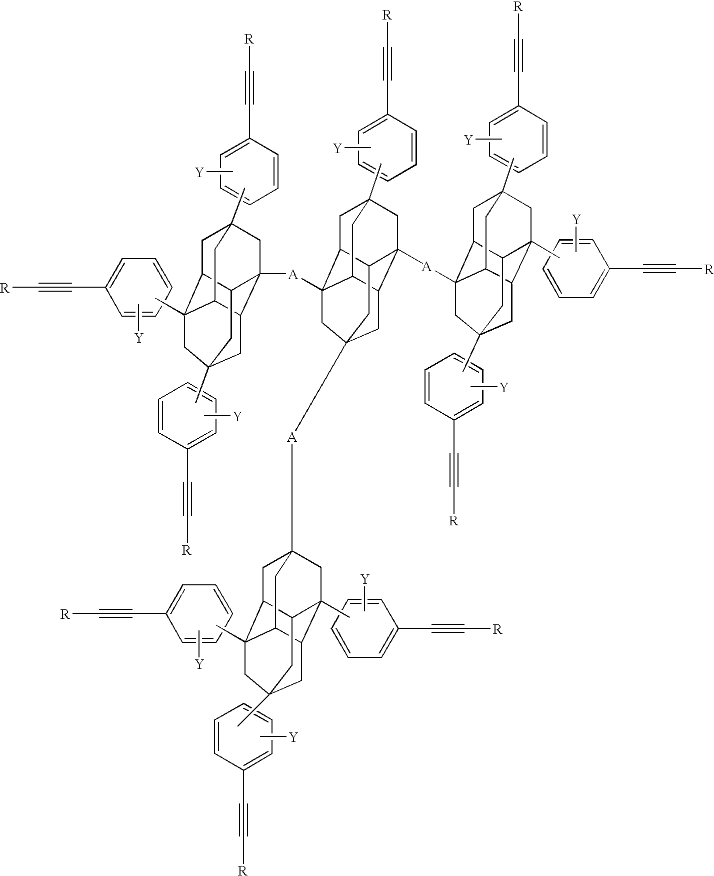

9. The composition of claim 1 wherein said oligomer or polymer is adamantane based.

10. The composition of claim 2 wherein said oligomer or polymer is adamantane based.

11. The composition of claim 2 wherein said A is Formula IV 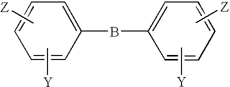
where B═(R₁)ₘ—(Ar)_{n—(C═C)}ₓ where m is 0, 1, 2, or 3; n is 0, 1, 2, 3, or greater; x is 2, 3, 4, or 5; R₁ is hydrogen, halogen, alkyl, t-butyl, phenyl, or substituted aryl; and Ar is phenyl, biphenyl, naphthyl, terphenyl, anthracenyl, polyphenylene, polyphenylene ether, or substituted aryl; Y is the same Y as above and Z is hydrogen, phenylethynyl, or the same as B above.

12. The composition of claim 11 wherein said B is (R₁)ₘ—(Ar)ₙ—(C≡C), where m is 0, 1, 2, or 3; n is 0, 1, 2, 3, or greater; x is 2, 3, 4, or 5; RI is hydrogen, halogen, alkyl, t-butyl, phenyl, or substituted aryl; and Ar is phenyl, biphenyl, naphthyl, terphenyl, anthracenyl, polyphenylene, polyphenylene ether, or substituted aryl.

13. The composition of claim 12 wherein said B is selected from the group consisting of m-diethynyl benzene; p-diethynyl benzene; diethynyl naphthalene; diethynyl biphenyl; and diethynyl phenylene oxide.

14. The composition of claim 11, further comprising adhesion promoter.

15. The composition of claim 11, further comprising porogen.

16. A spin-on composition comprising the composition of claim 2.

17. The spin-on composition of claim 16, further comprising at least one solvent.

18. A layer comprising the spin-on composition of claim 16.

19. The layer of claim 18, wherein the composition is cured.

20. A substrate having thereon the layer of claim 18.

21. A microchip comprising the substrate of claim 20.

22. A dielectric film comprising an irregular organic material having a thickness of up to about 25,000 Angstroms.

23. The dielectric film of claim 22, wherein the thickness is up to about 16,000 Angstroms.

24. The dielectric film of claim 23, wherein the thickness is up to about 10,000 Angstroms.

25. The dielectric film of claim 24, wherein the thickness is up to about 5,000 Angstroms.

26. The dielectric film of claim 25, wherein the thickness is up to about 1,000 Angstroms.

27. A method of forming a coating solution, comprising:
providing at least one of the compositions of claims 1 or 2;
providing at least one solvent; and
combining the at least one composition and the at least one solvent to form the coating solution.

28. The method of claim 27, further comprising providing at least one adhesion promoter and combining the at least one adhesion promoter with the at least one composition and the at least one solvent.

29. The method of claim 27, further comprising providing at least one porogen and combining the at least one porogen with the at least one composition and the at least one solvent.

30. The method of claim 28, further comprising providing at least one porogen and combining the at least one porogen with the at least one composition, the at least one adhesion promoter and the at least one solvent.
